(19) United States
(12) Patent Application Publication
(10) Pub. No.: US 2004/0127845 A1
(43) Pub. Date: Jul. 1, 2004

(54) Breast pump system

(75) Inventors: Charles J. Renz, Briarcliff Manor, NY (US); David Robson, Riverside, RI (US); Marco Wo, Providence, RI (US); Christopher Kampf, Cranston, RI (US); Aidan Petrie, Jamestown, RI (US); Michael Pereira, Smithfield, RI (US)

Correspondence Address: CHARLES N.J. RUGGIERO, ESQ., OHLANDT, GREELEY, RUGGIERO & PERLE, L.L.P., 10th FLOOR, ONE LANDMARK SQUARE, STAMFORD, CT, 06901-2682, US

(73) Assignee: Playtex Products, Inc.

(21) Appl. No.: 10/637,979

(22) Filed: Aug. 8, 2003

Related U.S. Application Data
(63) Continuation-in-part of application No. 10/331,183, filed on Dec. 27, 2002.
(63) Continuation-in-part of application No. 10/453,261, filed on Jun. 3, 2003.

(51) Int. Cl. A61M 1/06

(52) U.S. Cl. 604/74

(57) ABSTRACT

A breast pump system for obtaining breast milk is provided. The system sealingly separates the air flow from the breast milk and uses a single air tube for both positive pressure and negative pressure to be applied to a woman's breast. The breast pump can have a piston/cylinder device for generating pressure that allows a user to control suction and cycle time.

### RELATED APPLICATION

This application is a continuation-in-part of copending U.S. application Ser. No. 10/331,183, filed Dec. 27, 2002, the disclosure of which is incorporated by reference herein in its entirety. This application is also a continuation-in-part of copending U.S. application Ser. No. 10/453,261, filed Jun. 3, 2003, the disclosure of which is also incorporated by reference herein in its entirety.

### BACKGROUND OF THE INVENTION

1. Field of the Invention

The present invention relates to apparatus and methods for obtaining breast milk. More particularly, the present invention relates to a breast pump system that can apply a positive pressure or a negative pressure to a breast to express breast milk.

2. Description of the Related Art

Breast pump systems for obtaining breast milk, both manually and automatically, are known in the art. Conventional systems use a vacuum source to generate a negative pressure or vacuum that is transmitted through tubing to a breast hood or cup that is placed on the breast. This conventional device and method uses a negative pressure on the breast to express the breast milk.

Such systems suffer from the drawback of applying only a vacuum source as negative pressure to the breast to induce the expression of breast milk. Moreover, such conventional systems suffer from the drawback of applying the negative pressure or force axially to the nipple, resulting in elongation and distention of the nipple in an axial direction that is both uncomfortable and inefficient for the expression of breast milk.

### SUMMARY OF THE INVENTION

It is an object of the present invention to provide a breast pump system for expressing milk that can apply a positive pressure or a negative pressure to a breast to express the milk.

It is another object of the present invention to provide such a system that supplies the positive and negative pressure from a single source.

It is still another object of the present invention to provide such a system that facilitates control of the positive and negative pressure applied to the breast.

It is yet another object of the present invention to provide such a system that widens the nipple to express milk.

It is a further object of the present invention to provide such a system that reduces axial elongation or distention of the nipple.

It is another further object of the present invention to apply a negative force or negative pressure gradient to the nipple that has a greater lateral component than axial component.

It is yet a further object of the present invention to accommodate breasts of differing size and/or shape by providing a kit with interchangeable breast hoods of differing size and/or shape.

It is still a further object of the present invention to substantially monitor and control the pressure source in real-time.

These and other objects and advantages of the present invention are provided by a breast cup having a hood for receiving the breast and in fluid communication with a pressure source. The hood creates a negative force on the nipple during a negative pressure stroke. The negative force has a lateral component and an axial component. The lateral component is greater than the axial component.

The present invention includes a breast cup having a breast receiving member in fluid communication with a vacuum source. The breast receiving member applies a negative pressure to the nipple during a negative pressure stroke causing the nipple to widen along a lateral direction.

The present invention includes a breast pump system having a pressure source and a breast cup for receiving the breast. The breast cup is in fluid communication with the pressure source. The breast cup creates a negative force on the nipple during a negative pressure stroke. The negative force has a lateral component and an axial component. The lateral component is greater than the axial component.

The present invention includes a breast pump system having a vacuum source and a breast receiving member that is in fluid communication with the vacuum source. The breast receiving member applies a negative pressure to the nipple during a negative pressure stroke causing the nipple to widen along a lateral direction.

The present invention includes a breast pump kit having a holder and a plurality of hoods for receiving the breast. Each of the plurality of hoods are selectively engageable to the holder and a pressure source for expressing the breast milk from the breast. At least one of the plurality of hoods has a different size or a different shape than another of the plurality of hoods.

The present invention includes a breast pump system having a pump generating pressure and a plurality of hoods for receiving the breast. Each of the plurality of hoods are selectively fluidly connectable to the pump for expressing the breast milk from the breast. At least one of the plurality of hoods has a different size or a different shape than another of the plurality of hoods.

The present invention includes a breast pump having a pressure source with a movable structure for generating pressure during a pressure stroke. The movable structure has a variable pressure volume or variable cycle time. The pump also has a controller operably connected to the pressure source. The controller regulates the pressure volume based upon a distance traveled by the movable structure or regulates the variable cycle time based upon a speed of the movable structure. The controller provides substantially real-time monitoring of the distance traveled or the speed.

The present invention includes a breast pump system having a pressure source with an evacuation volume for generating a pressure and an air hole. The system also has a breast cup for receiving the breast and in fluid communication with the pressure source for applying the pressure to the breast. The air hole has a diameter and is in fluid communication with the atmosphere and the evacuation volume. The diameter of the air hole is between about 0.15 mm to 0.75 mm.

The present invention includes a method of expressing breast milk from a breast having the steps of applying a negative pressure to the breast from a pressure source during a vacuum stroke; applying a positive pressure to the breast from the pressure source during a massage stroke; and providing air from the atmosphere to the pressure source during the vacuum stroke.

The present invention includes a method of expressing breast milk from a breast having the step of applying a negative pressure on at least a portion of the nipple causing the nipple to widen along a lateral direction.

The present invention includes a method of expressing breast milk from a breast having the steps of applying a pressure to the breast; and performing substantially real-time monitoring and controlling of the pressure with a controller.

The present invention includes a pump for providing pressure that has a housing, an actuator and an insert. The housing defines a volume and has a pressure exhaust. The actuator is operably connected to the housing for producing the pressure in the volume. The insert is connected to the housing. The insert has a hole disposed therethrough. The hole provides fluid communication between the volume and atmosphere.

The present invention includes a breast cup for placing a breast in fluid communication with a first container and a second container that have openings with different diameters. The breast cup has a funnel for receiving the breast and a housing connected to the funnel. The funnel has a base. The base has a circumferential wall, a flange extending inwardly from the circumferential wall to define an opening, first threads, and second threads. The first threads have a first diameter and a first pitch. The first diameter and the first pitch allow for selective engagement with the first container. The second threads have a second diameter and a second pitch. The second diameter and the second pitch allow for selective engagement with the second container. The first threads and the second threads are concentrically disposed along the base.

The present invention includes a nipple ring for engaging a nipple with a first container and a second container that have openings with different diameters. The nipple ring has a body having a circumferential wall, a flange extending inwardly from the circumferential wall to define an opening, first threads, and second threads. The first threads have a first diameter and a first pitch. The first diameter and the first pitch allow for selective engagement with the first container. The second threads have a second diameter and a second pitch. The second diameter and the second pitch allow for selective engagement with the second container. The first threads and the second threads are concentrically disposed along the body.

The present invention includes a cap for engaging with a first container and a second container that have openings with different diameters. The cap has a body with a circumferential wall, a top wall connected to the circumferential wall, first threads, and second threads. The first threads have a first diameter and a first pitch. The first diameter and the first pitch allow for selective engagement with the first container. The second threads have a second diameter and a second pitch. The second diameter and the second pitch allow for selective engagement with the second container. The first threads and the second threads are concentrically disposed along the body.

The first pitch can be equal to the second pitch. The first threads can extend from the flange. The second threads can be disposed on the circumferential wall. The funnel can be selectively removable from the housing.

The housing can be a first material and the insert can be a second material. The housing can be plastic and the insert can be metal. The housing can be a cylinder and the actuator can be a piston. The cylinder can be an orifice and the insert can be disposed in the orifice. The insert can be press fit into the orifice. The insert can be a plurality of inserts, and each of the plurality of inserts can be selectively engageable with the cylinder.

The breast cup can also have a barrier member operably connected to the hood, wherein the barrier member reduces the axial component of the negative force during the negative pressure stroke. The hood can have a housing, a flexible insert sealingly secured to the housing, and a displacement volume formed between the housing and the flexible insert, wherein the displacement volume is in fluid communication with the pressure source. The displacement volume can substantially surround the nipple when the breast is received in the hood. The flexible insert can have a bladder in fluid communication with the pressure source with the displacement volume being defined at least partially by the bladder. The bladder and the displacement volume can contract to form the negative force on the nipple during the negative pressure stroke.

The breast cup can also have a barrier member disposed substantially adjacent to the bladder, thereby preventing the breast from contacting the bladder. The flexible insert can define an inner volume for receiving the breast, and the barrier member can have a cylindrical shape and be disposed in the inner volume. The flexible insert can have a funnel shape with a massaging projection formed thereon. The massaging projection can have a star-like shape.

The negative pressure created at the breast cup can cause the nipple to widen along a lateral direction more than the nipple elongates along an axial direction. The negative pressure can have an average lateral component and an average axial component, wherein during the negative pressure stroke the average lateral component is greater than the average axial component. The barrier member can be operably connected to the breast receiving member, and can reduce elongation of the nipple along the axial direction during the negative pressure stroke. The breast receiving member can have a housing, a flexible insert sealingly secured to the housing, and a displacement volume formed between the housing and the flexible insert, wherein the displacement volume is in fluid communication with the vacuum source.

The vacuum or pressure source can be a piston movably disposed in a cylinder. The system can have a reversible motor operably connected to the piston. The system can also have a rack having first teeth and a gear having second teeth. The rack can be connected to the piston and the gear can be operably connected to the reversible motor. The first teeth can engage with the second teeth to reciprocally move the piston in the cylinder. The cylinder can have a first diameter and an air hole. The air hole can have a second diameter and be in fluid communication with the atmosphere. The first diameter of the cylinder can be significantly larger than the second diameter of the air hole.

The system can have a controller operably connected to the reversible motor. The controller can determine a distance that the piston has traveled relative to the cylinder. The controller can reverse the motor based at least in part upon that distance. The system can also have a motor with variable speed. The controller can adjust the speed based upon a desired cycle time for applying the negative pressure to the breast. The controller can regulate the pressure cycle based on a non-sinusoidal wave signal of pressure versus variable cycle time.

Each of the plurality of hoods of the kit can have a housing, a flexible insert sealingly secured to the housing, and a displacement volume formed between the housing and the flexible insert and in fluid communication with the pressure source. The housing and/or the flexible insert of the at least one of the plurality of hoods can have a different size or a different shape than the housing and/or the flexible insert of the another of the plurality of hoods. The kit can also have a container, wherein the holder is selectively engageable with the container. The holder can have a plurality of engagement structures for selectively engaging a plurality of different sized containers. The flexible insert of the at least one of the plurality of hoods can have a first massaging projection, and the flexible insert of the another of the plurality of hoods can have a second massaging projection. The first and second massaging projections can have a different size or a different shape.

### BRIEF DESCRIPTION OF THE DRAWINGS

Other and further objects, advantages and features of the present invention will be understood by reference to the following:

FIG. 1 is a front perspective view of a breast pump of the breast pump system of the present invention;

FIG. 2 is a front perspective view of the breast pump of FIG. 1 in an opened position;

FIG. 3 is an exploded perspective view of the breast pump of FIG. 1;

FIG. 4 is a top view of the breast pump of FIG. 1 without the cover;

FIG. 5 is an exploded perspective view of a piston and cylinder of the present invention;

FIG. 6 is an exploded side view of a portion of the piston and cylinder of FIG. 5;

FIG. 7 is a front perspective view of the piston of FIG. 5;

FIG. 8 is an exploded perspective view of an alternative embodiment of the piston of the present invention;

FIG. 9 is an exploded perspective view of a pressure relief valve of the system of FIG. 1;

FIG. 10 is a cross-sectional plan view of the cylinder of FIG. 5;

FIG. 11 is a front perspective view of a breast cup of the present invention;

FIG. 12 is a side cross-sectional view of the breast cup of FIG. 11;

FIG. 13 is a rear perspective view of a T-connector of the present invention;

FIG. 14 is a flow chart depicting a method for pumping a breast according to the system of FIGS. 1 and 11;

FIG. 15 is a top perspective view of a preferred embodiment of breast pump for the breast pump system of the present invention;

FIG. 16 is a top view of the breast pump of FIG. 15;

FIG. 17 is a top perspective view of the drive system of the breast pump of FIG. 15;

FIG. 18 is a side perspective view of the drive system of FIG. 17;

FIG. 19 is a top perspective view of a portion of the gear reduction system of the drive system of FIG. 15, partially assembled;

FIG. 20 is a top perspective view of an alternative embodiment of breast pump for the breast pump system of the present invention;

FIG. 21 is a top view of the breast pump of FIG. 20;

FIG. 22 is a top perspective view of the drive system of the breast pump of FIG. 20;

FIG. 23 is a side perspective view of the drive system of FIG. 20;

FIG. 24 is a top perspective view of the motor of the drive system of FIG. 20;

FIG. 25 is a top perspective view of a portion of the gear reduction system of the drive system of FIG. 20, partially assembled;

FIG. 26 is a top perspective view of the gear reduction system of the drive system of FIG. 20, partially assembled;

FIG. 27 is a partial cross-sectional side view of the breast cup of FIG. 11 with a breast;

FIG. 28 is a partial cross-sectional side view of the breast cup of FIG. 27 applied to the breast prior to the negative pressure stroke;

FIG. 29 is an exploded cross-sectional view of the breast cup and breast of FIG. 27 during the negative pressure stroke showing a representation of the negative pressure gradient or force on the breast;

FIG. 30 is a cross-sectional side view of a prior art breast cup applied to a breast prior to the negative pressure stroke;

FIG. 31 is an exploded cross-sectional view of the prior art breast cup and breast of FIG. 30 during the negative pressure stroke showing a representation of the negative pressure gradient or force on the breast;

FIG. 32 is an exploded perspective view of the pressure relief valve of FIG. 9 with another embodiment of a relief assembly;

FIG. 33 is a perspective view of the cylinder of FIG. 5 with another embodiment of the pressure differential hole;

FIG. 34 is an alternative embodiment of a breast cup of the present invention;

FIG. 35 is another alternative embodiment of a breast cup of the present invention;

FIG. 36 is a bottom perspective view of a nipple ring with a nipple of the present invention;

FIG. 37 is a side cross-sectional view of the nipple ring and nipple of FIG. 36;

FIG. 38 is a bottom perspective view of a cap of the present invention; and

FIG. 39 is a side cross-sectional view of the cap of FIG. 38.

### DESCRIPTION OF THE INVENTION

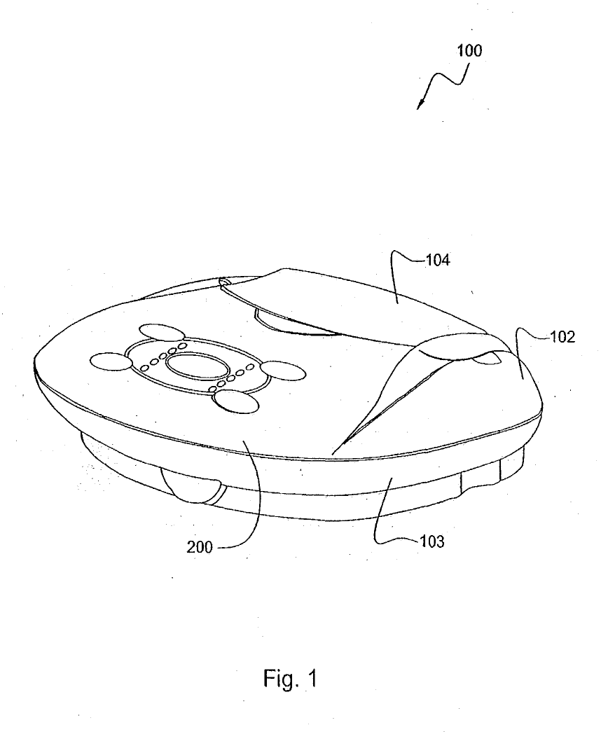

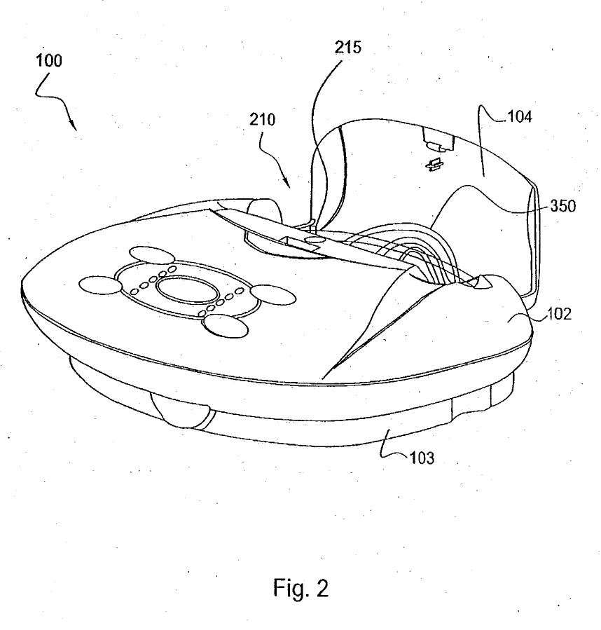

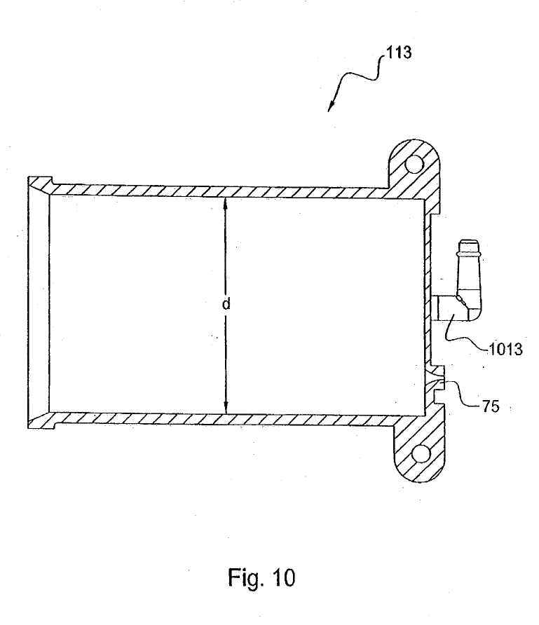

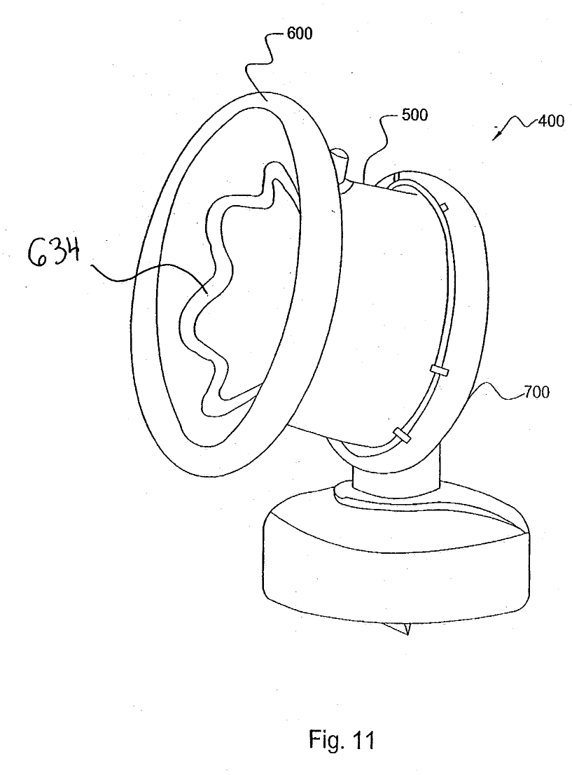

Referring to the drawings and, in particular, FIGS. 1 and 2, there is shown a breast pump of the present invention generally represented by reference numeral 100. Breast pump 100, along with breast cup 400 shown in FIG. 11, form the major components of the breast pump system of the present invention. Breast pump 100 has a top housing 102 and a bottom housing 103 that are adapted to form an assembled unit.

Referring to FIGS. 1 through 3, top housing 102 has a substantially ellipsoidal shape with a flat front face 200 and a storage compartment 210 having a compartment door 104. Preferably, door 104 is hingedly connected to top housing 102 to form a selectively sealable storage compartment 210 for storing air tubing or conduit 350 that connects breast pump 100 to the other components of the system, which will be discussed later in greater detail.

Face 200 can receive a button pad 105 having an LED cover 106. Pad 105 is used by the consumer to control breast pump 100. Bottom housing 103 can securely house the various components of the breast pump, which include a rack gear 109, a pinion gear 110 that can engage the rack gear, a piston 112, a cylinder 113 that can receive the piston, and a motor 125 having a shaft 126 upon which the pinion gear is mounted. Due in part to this design, breast pump 100 provides pumping with low noise. Breast pump 100 can be made of any rigid material, such as, for example, plastic.

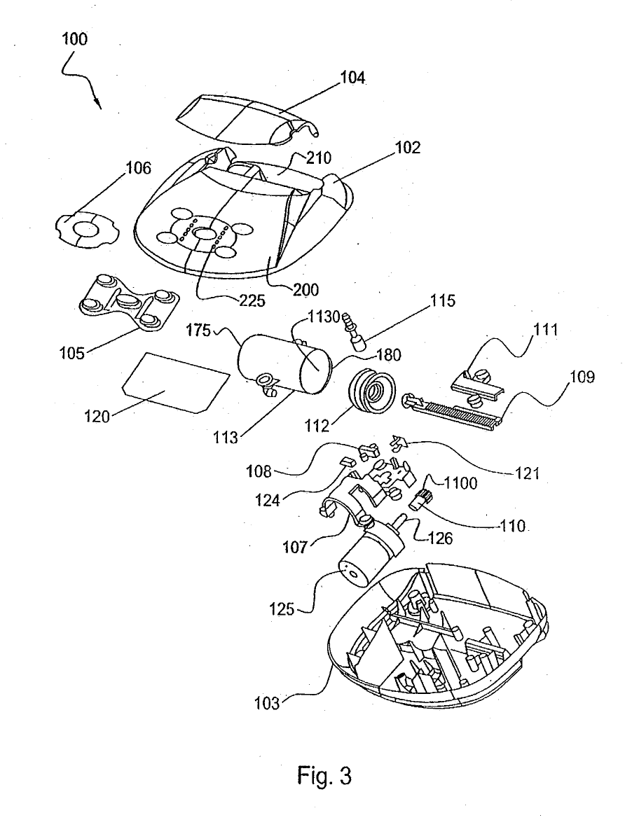

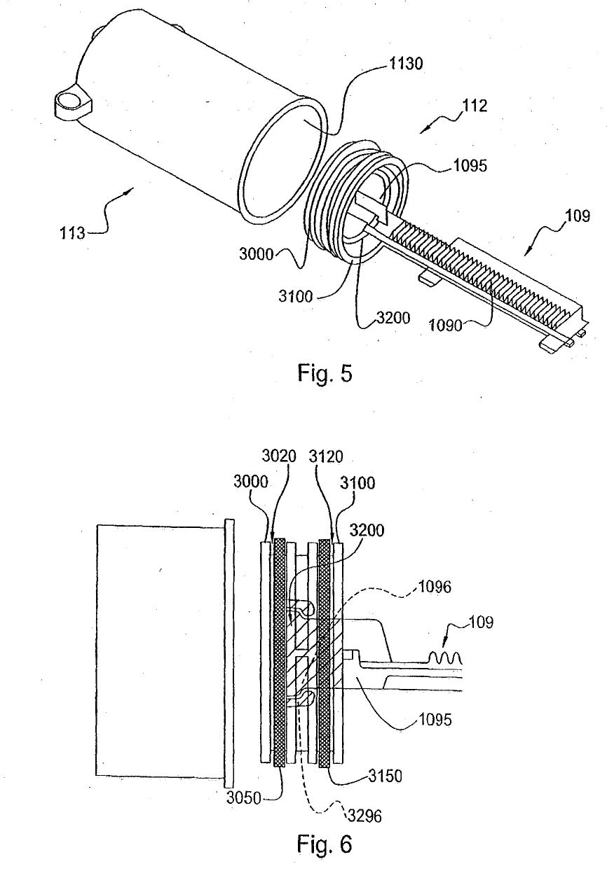

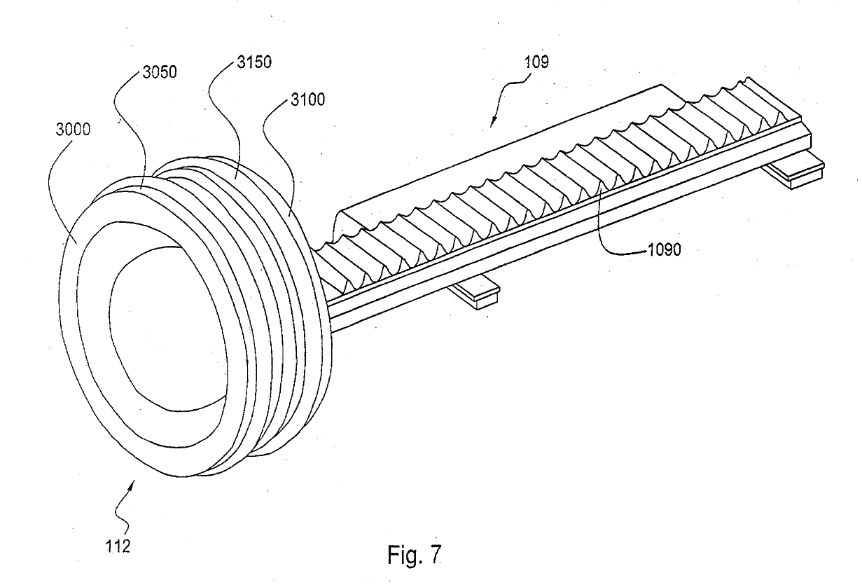

Referring to FIGS. 3 through 7, breast pump 100 utilizes piston 112 and cylinder 113 to create both a positive pressure and a negative pressure for obtaining breast milk. Piston 112 is driven by rack gear 109, which is affixed thereto. Piston 112 has a substantially cylindrical-shape with a first head 3000 and a second head 3100. First and second heads 3000, 3100 preferably have annular channels 3020, 3120 formed therein, respectively. Channels 3020, 3120 are disposed along the outer circumference of first and second heads 3000, 3100, respectively. Preferably, channels 3020, 3120 are centrally located along the outer circumference of first head 3000 and second head 3100. Seated in channels 3020, 3120 are sealing members 3050, 3150, respectively. Preferably, sealing members 3050, 3150 are o-ring gaskets. Sealing members 3050, 3150 have a diameter or width that is larger than the depth or height of channel 3020 and channel 3120. Sealing members 3050, 3150 extend beyond the outer circumference of first head 3000 and second head 3100 forming a sealing engagement with an inner surface 1130 of cylinder 113 as piston 112 is driven back and forth in the cylinder.

The use of multiple sealing members, i.e., o-ring gasket 3050 and o-ring gasket 3150 on piston 112, provide a double sealing to increase the efficiency of creating the positive pressure and negative pressure. While this embodiment uses two sealing members to create two separate sealing surfaces, any number of sealing members can be used to create any number of sealing surfaces for sealing piston 112 with cylinder 113. Additionally, while this embodiment uses piston 112 having o-ring sealing gaskets 3050, 3150, alternative sealing structures can be used between the piston and cylinder 113.

Rack gear 109 has teeth 1090 that engage with pinion gear 110 having teeth 1100. Pinion gear 110 is operatively connected to motor 125, preferably via shaft 126. When motor 125 is activated, shaft 126 and pinion gear 110 rotate. Teeth 1090 on rack 109 and teeth 1100 on pinion 110 mesh and translate the reciprocal rotational motion of motor 125 and shaft 126 into a reciprocal longitudinal motion along a single axis in both directions.

Preferably, rack gear 109 has a first end 1095 that engages with a recess 3200 formed in piston 112. Recess 3200 is preferably centrally located in piston 112. First end 1095 of rack gear 109 preferably has a snap fit or friction fit engagement with recess 3200 of piston 112. Preferably, there are detent structures 1096, 3296 formed on first end 1095 and recess 3200, respectively. This facilitates production of these components and also provides for any slight pivotal movement that may be required of piston 112 with respect to rack gear 109.

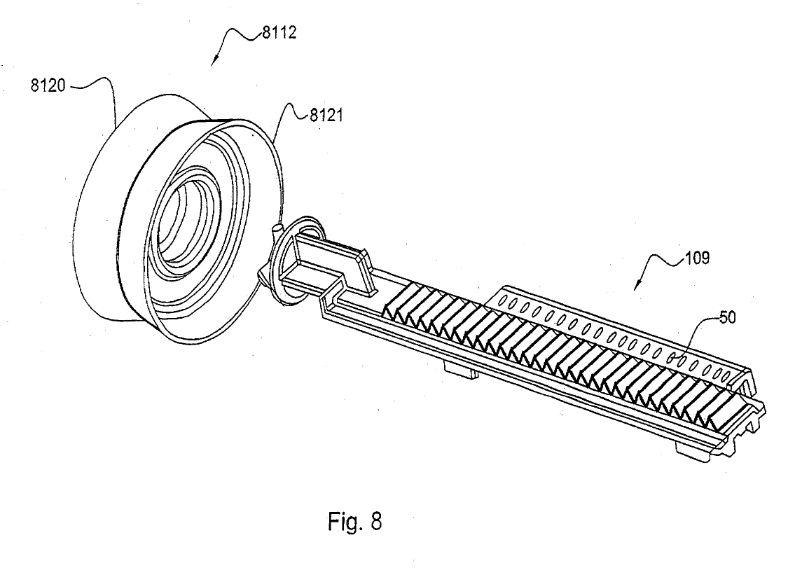

An alternative embodiment of a piston is shown in FIG. 8 and generally represented by reference numeral 8112. Piston 8112 has a substantially V-shape with a leading edge 8120 and a trailing edge 8121. Leading edge 8120 and trailing edge 8121 sealingly engage an inner surface 1130 of cylinder 113 as piston 8112 is driven back and forth in the cylinder. The use of multiple edges, i.e., leading edge 8120 and following edge 8121, on piston 8112 that sealingly engage inner surface 1130 of cylinder 113, provide a double sealing to increase the efficiency of creating the positive pressure and negative pressure.

Referring to FIGS. 3 through 7, motor 125 is preferably variable speed. This allows a user to control and vary the cycle time of the pumping of the breast. Breast pump 100 further has a motor cover 107 and a bearing 108 to reduce vibration and to secure motor 125 to bottom housing 103.

The positive and negative pressures can be varied by changing the displacement of air volume in cylinder 113. In this embodiment, this is done by use of a photoelectric or photo-sensor system. The photo-sensor system has two or more photo-sensors 121 and a position switch 124. The photo-sensors 121 count the number of openings 50 on rack gear 109, as the rack gear moves back and forth. Thus, a user can control the distance that rack gear 109 travels and correspondingly control the air volume displacement in cylinder 113. Alternative displacement or distance monitors can also be used, such as, for example a coded wheel for counting the slots on the wheel; counting of the belt teeth; rotary encoder which counts its own revolutions; or a hall effect sensor.

To ensure that piston 112 is properly moving to the front of cylinder 113, the photo-sensor system further includes position switch 124, preferably located at the front of the cylinder, which acts as a starter for the counter. Alternatively, the position switch can be an opening 50 having a different size or shape that is detectable by photo-sensor 121.

Rack gear 109 can also have a safety mechanism attached thereto. Photo-sensor 121 will be reading openings 50 as rack gear 109 moves backwards. If for some reason rack gear 109 misses its target and moves too far, the safety will trigger the position switch. When the position switch is triggered while rack gear 109 is moving backwards, the software can trigger the system to move forward again and return to the position position.

Breast pump 100 has a guide cover 111 positioned over rack gear 109. Guide cover 111 provides added stability to the breast pump by guiding and vibration dampening the reciprocal movement of rack gear 109. Guide cover 111 also provides accuracy to the photo-sensor system by reducing the risk of misalignment of photo-sensors 121 and openings 50.

The photo-sensor system and motor 125 are preferably connected to a PC or circuit board 120. Thus, the distance piston 112 travels, which translates to the amount of positive and negative pressure, and the piston speed, which translates to the cycle time, are electronically controlled.

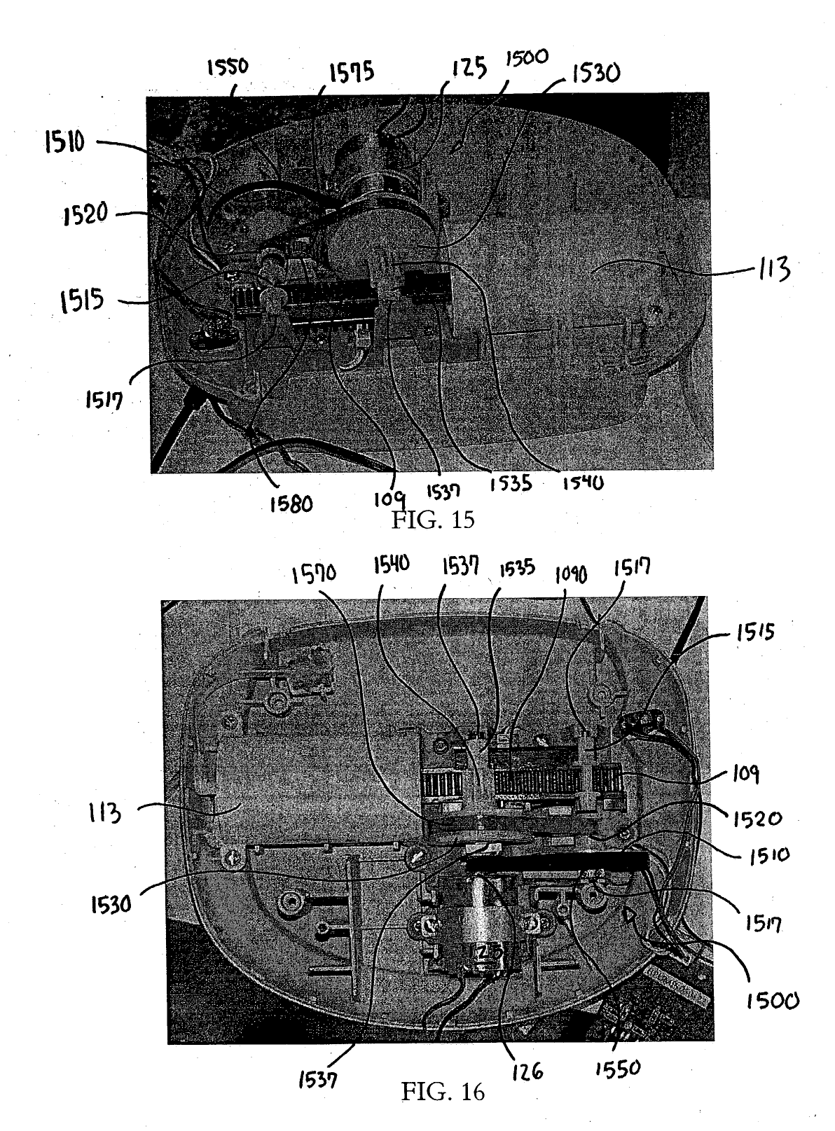

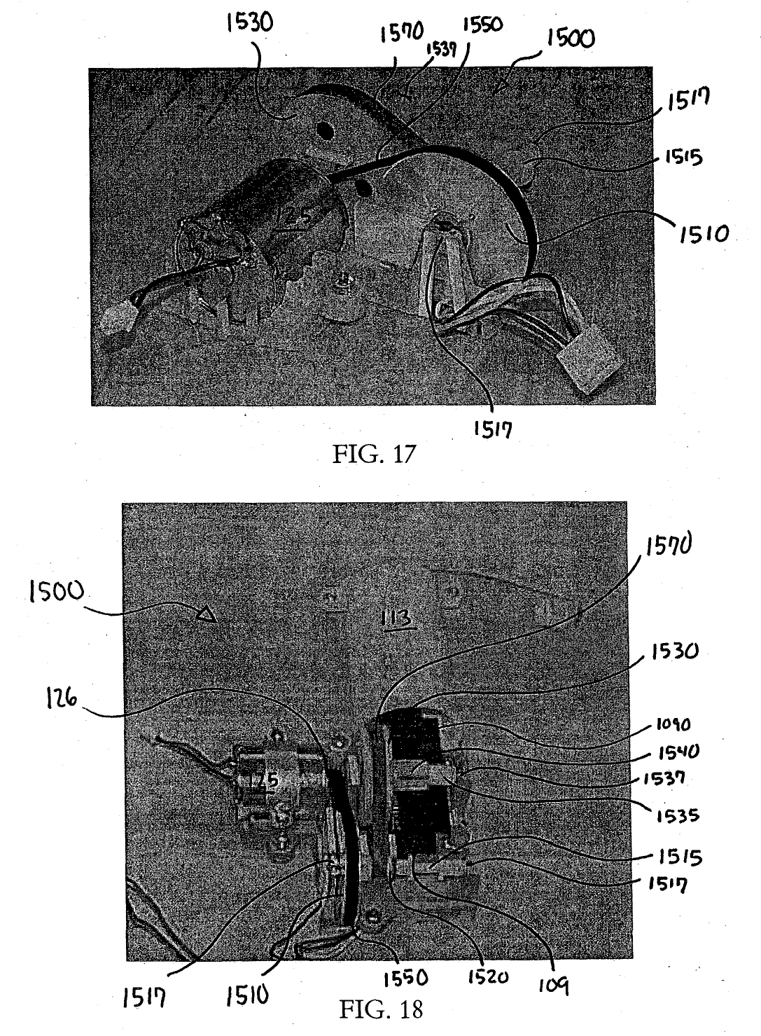

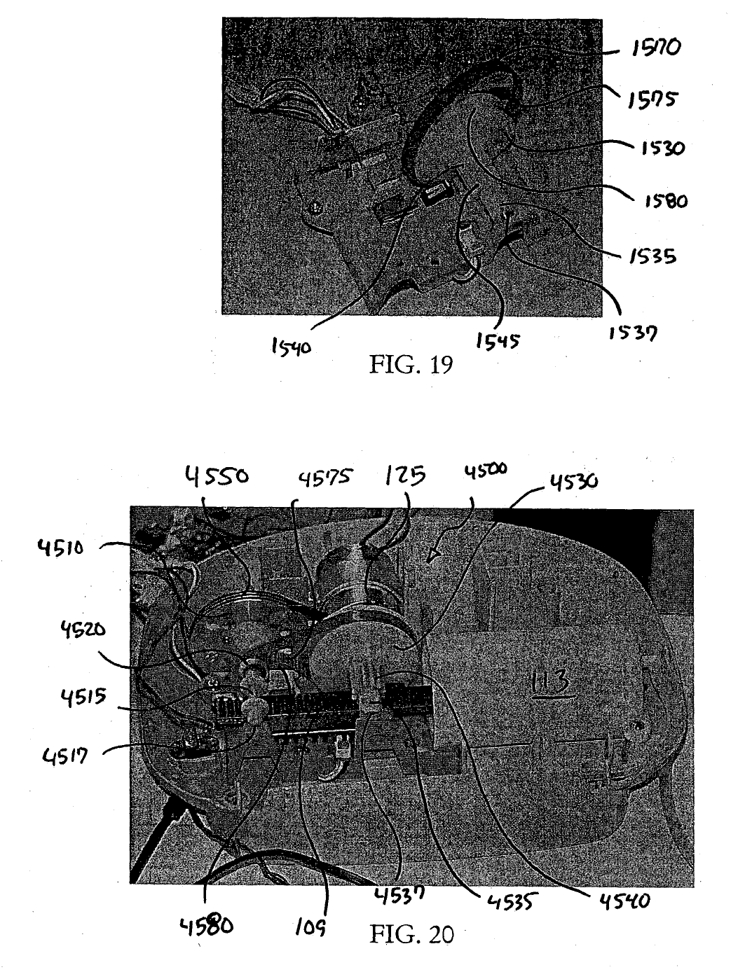

Referring to FIGS. 15 through 19, a preferred embodiment of a drive system of the present invention is shown and generally represented by reference numeral 1500. Drive-system 1500 is usable with breast pump 100 of FIGS. 1 through 7 to provide the linear reciprocal movement of piston 112 with cylinder 113.

Drive system 1500 is a belt drive system for a rack and pinion drive having gear reduction incorporated therein. Drive system 1500 has a first drive wheel or pulley 1510; a second gear, drive wheel or pulley 1520 secured to the first drive wheel 1510; a third gear, drive wheel or pulley 4530; and a pinion gear 1540 secured to the third gear.

First drive wheel 1510 is operably connected to motor drive shaft 126 by a first belt 1550. In the preferred embodiment, first belt 1550 is a non-toothed belt. More preferably, first belt 1550 has resiliency or flexibility. The use of flexible or resilient belt 1550 provides a secure connection between drive shaft 126 and first drive wheel 1510 and also reduces noise and vibration. Drive shaft :126 and first drive wheel 1510 have smooth outer surfaces upon which the first belt 1550 is secured.

First drive wheel 1510 is operably connected to second gear,1520 by a first co-axial shaft 1515. In the preferred embodiment, first shaft 1515 is rotatably mounted between opposing first bearings 1517. However, alternative rotatable mounting arrangements or securing structures could also be used. To reduce noise and vibration, motor shaft 126 and first drive wheel 1510 are made of metal. First drive wheel 1510 and second gear 1520 have different diameters that partially provide for gear reduction between motor shaft 126 and pinion gear 1540.

Second gear 1520 is operably connected to third gear 1530 by a second belt 1570. Preferably, second belt 1570 has teeth 1575 that mesh with teeth 1580 formed along the circumference of second gear 1520 and third gear 1530. Second and third gears 1520, 1530 have different diameters that partially provide for gear reduction between motor shaft 126 and pinion gear 1540. Drive system 1500 can also have a tension pulley 1580 that provides tension to second belt 1570.

Third gear 1530 is operably connected to pinion gear 1540 by a second co-axial shaft 1535. In the preferred embodiment, second shaft 1535 is rotatably mounted between opposing second bearings 1537. However, alternative rotatable mounting arrangements or securing structures could also be used. Preferably, third gear 1530 is integrally molded with pinion gear 1540 along second shaft 1535.

Pinion gear 1540 has teeth 1545 that engage with teeth 1090 of rack gear 109. When motor 125 is activated, the rotational motion of shaft 126 is translated into a reciprocal longitudinal motion along a single axis of rack gear 109 in both directions. Drive system 1500, through use of first and second belts 1550, 1570 and first, second and third drive wheels or gears 1510, 1520, 1530, is able to provide a desired ratio of movement between motor shaft 126 and pinion gear 1540, i.e., gear reduction.

The use of a combination of the non-toothed belt 1550 and the toothed belt 1570 reduces noise and vibration, while maintaining a secure, sturdy drive system 1500 that is able to provide the necessary back and forth linear motion at the desired speeds and pressure for breast pump 100.

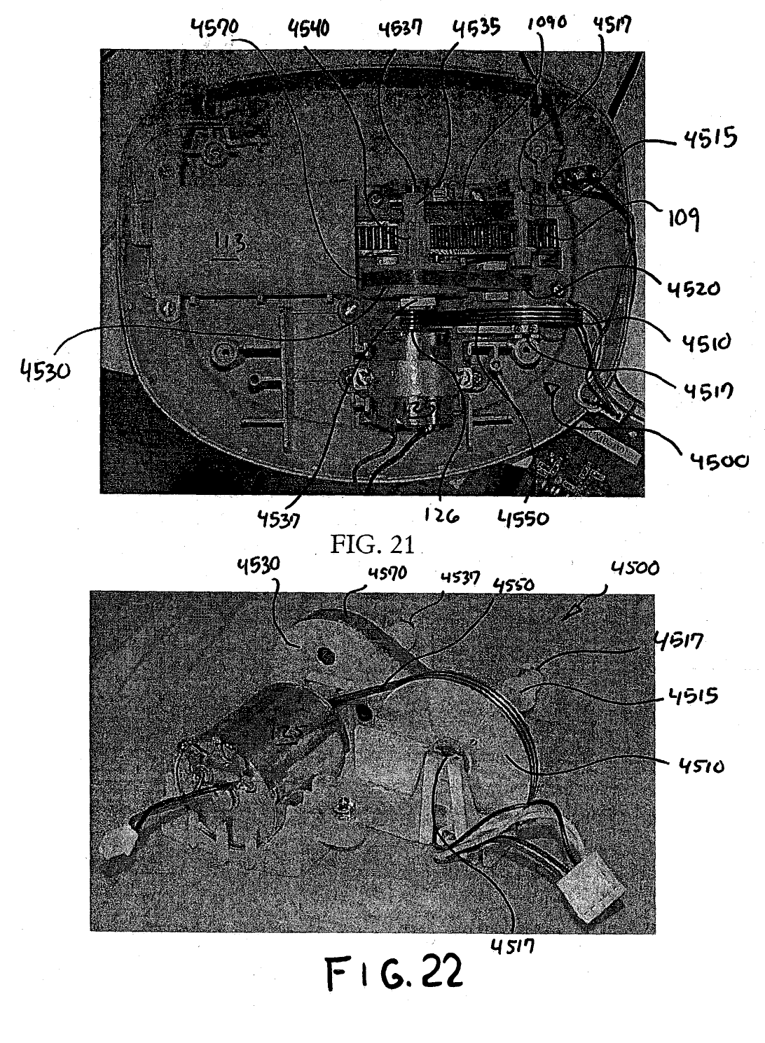

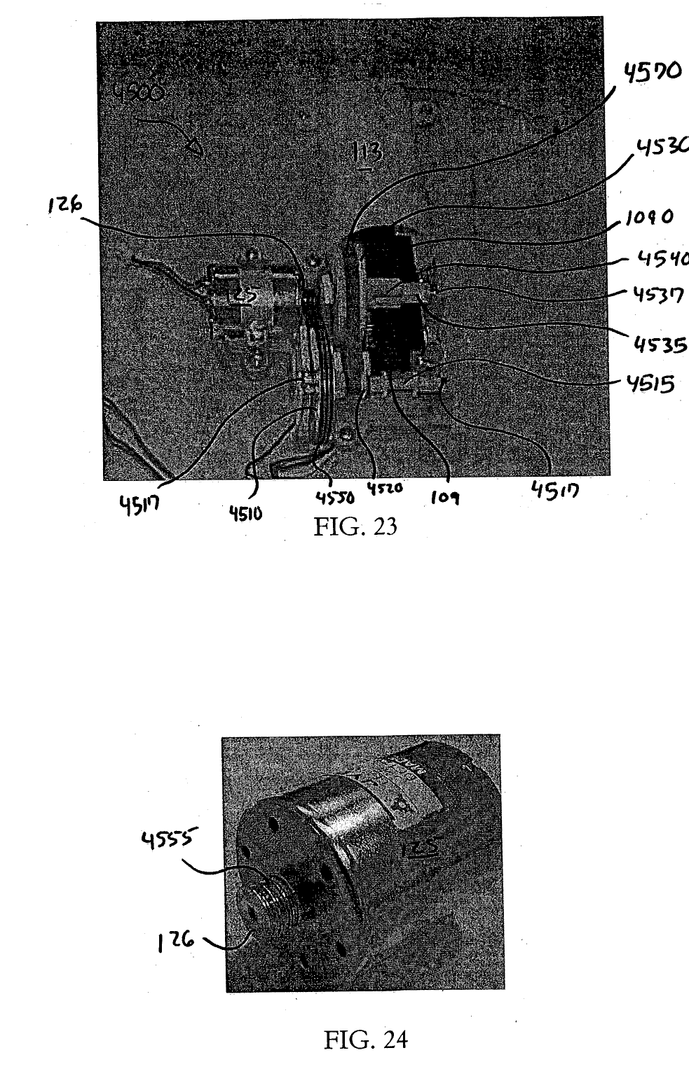

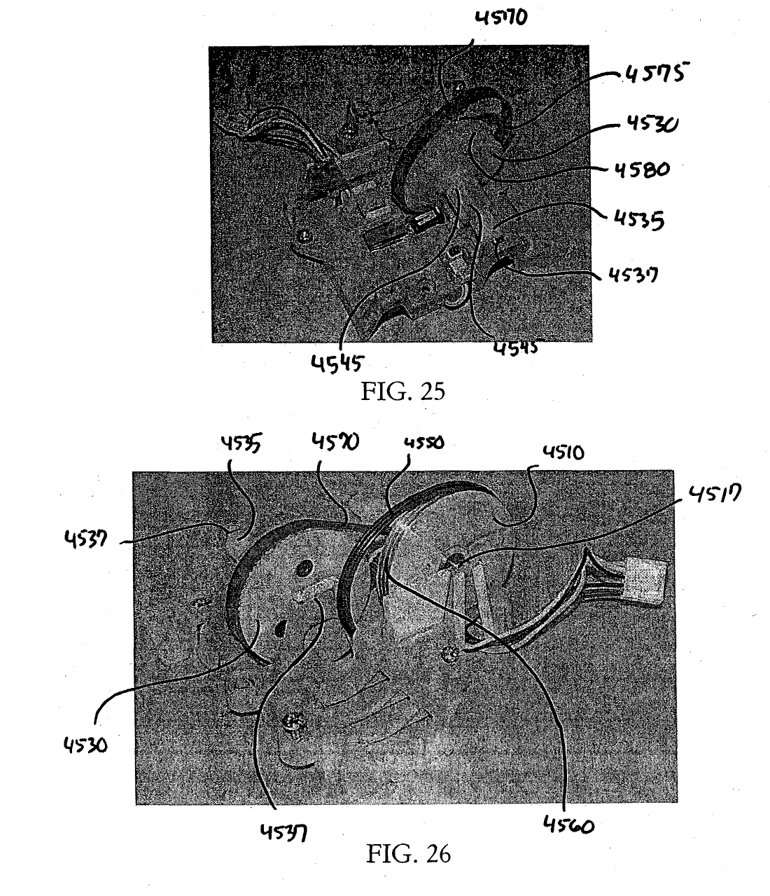

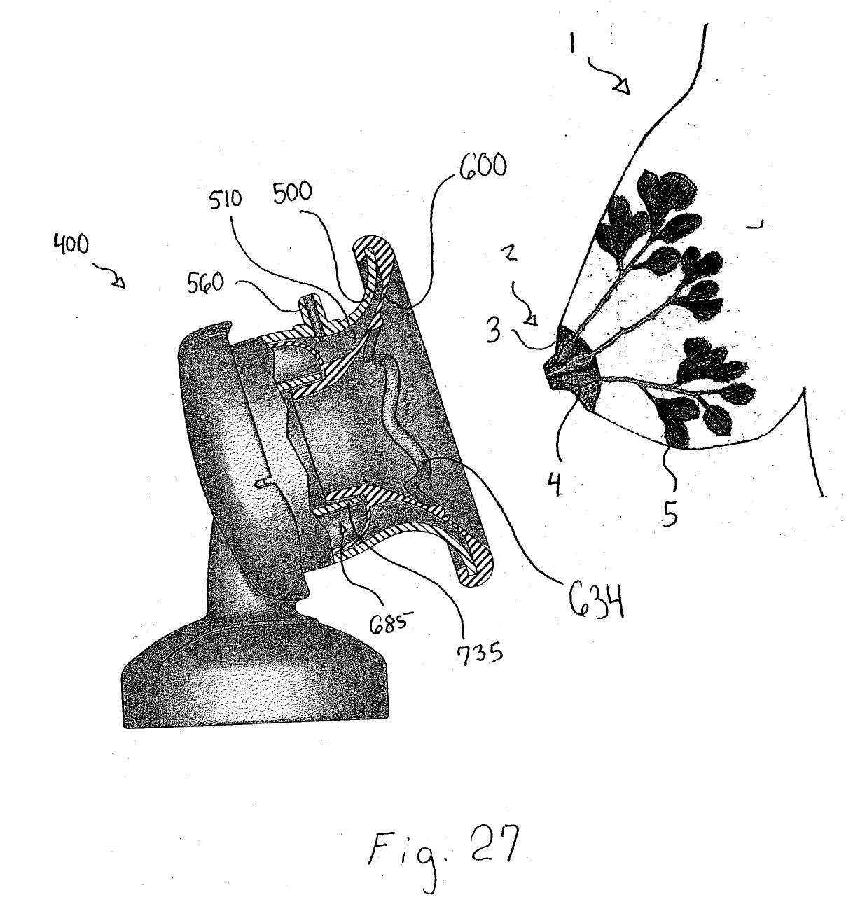

Referring to FIGS. 20 through 26, an alternative embodiment of a drive system of the present invention is shown and generally represented by reference numeral 4500. Drive system 4500 is also usable with breast pump 100 of FIGS. 1 through 7 to provide the linear reciprocal movement of piston 112 with cylinder 113.

Drive system 4500 is a belt drive system having gear reduction incorporated therein. Drive system 4500 has a first gear, drive wheel or pulley 4510; a second gear, drive wheel or pulley 4520 secured to the first gear; a third gear, drive wheel or pulley 4530; and a pinion gear 4540 secured to the third gear.

First gear 4510 is operably connected to motor drive shaft 126 by a first belt 4550. In the preferred embodiment, first belt 4550 is a plurality of belts, and more preferably, three belts. First belts 4550 are preferably non-toothed belts. More preferably, first belts 4550 are o-rings having resiliency or flexibility. The use of flexible or resilient belts 4550, such as, for example, o-rings, provides a secure connection between drive shaft 126 and first gear 4510, and also reduces noise and vibration. Drive shaft 126 and first gear 4510 have annular channels 4555, 4560, formed therein, respectively. Annular channels 4555, 4560 are guides that assist in holding first belts 4550 in place and facilitate assembly of drive system 4500.

First gear 4510 is operably connected to second gear 4520 by a first co-axial shaft 4515. In this alternative embodiment, first shaft 4515 is rotatably mounted between opposing first bearings 4517. However, alternative rotatable mounting arrangements or securing structures could also be used. To reduce noise and vibration, motor shaft 126 and first gear 4510 are made of metal. First and second gears 4510, 4520 have different diameters that partially provide for gear reduction between motor shaft 126 and pinion gear 4540.

Second gear 4520 is operably connected to third gear 4530 by a second belt 4570. Preferably, second belt 4570 has teeth 4575 that mesh with teeth 4580 formed along the circumference of second gear 4520 and third gear 4530. Second and third gears 4520, 4530 have different diameters that partially provide for gear reduction between motor shaft 126 and pinion gear 4540. Drive system 4500 can also have a tension pulley 4580 that provides tension to second belt 4570.

Third gear 4530 is operably connected to pinion gear 4540 by a second co-axial shaft 4535. In this alternative embodiment, second shaft 4535 is rotatably mounted between opposing second bearings 4537. However, alternative rotatable mounting arrangements or securing structures could also be used. Preferably, third gear 4530 is integrally molded with pinion gear 4540 along second shaft 4535.

Pinion gear 4540 has teeth 4545 that engage with teeth 1090 of rack gear 109. When motor 125 is activated, the rotational motion of shaft 126 is translated into a reciprocal longitudinal motion along a single axis of rack gear 109 in both directions. Drive system 4500, through use of first and second belts 4550, 4570 and first, second and third gears 4510, 4520, 4530, is able to provide a desired ratio of movement between motor shaft 126 and pinion gear 4540, i.e., gear reduction.

The use of a combination of the non-toothed o-ring belts 4550 and the toothed belt 4570 reduces noise and vibration, while maintaining a secure, sturdy drive system 4500 that is able to provide the necessary back and forth linear motion at the desired speeds and pressure for breast pump 100.

The embodiments of the drive systems 1500 and 4500 described above utilize belts for gear reduction. However, alternative embodiments can use a gear-box that reduces the gearing to the desired ratio that is transferred to the rack and pinion gearing that drives breast pump 100.

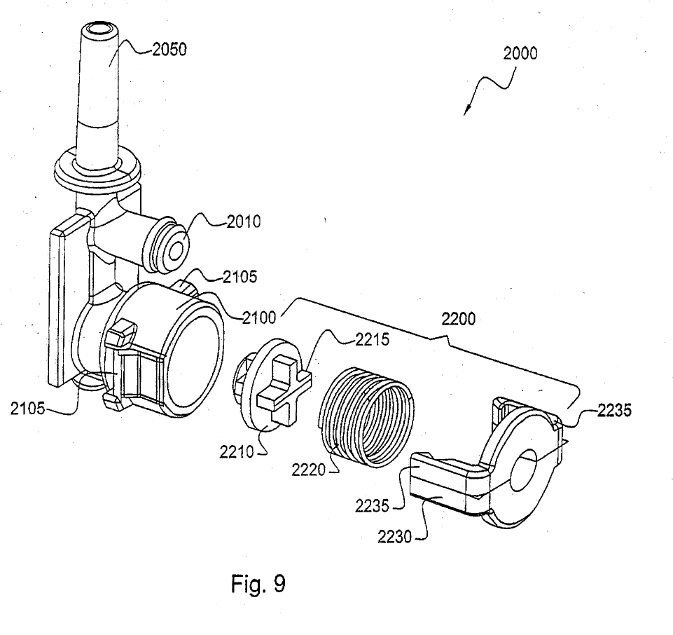

Referring back to FIGS. 3 through 9, cylinder 113 has a supply tube 116 that is secured to a supply connector 115 for supplying the positive and negative pressure to breast cup 400. Preferably, supply connector has an outlet 215 disposed in storage compartment 210. Air tubing 350 can be secured to outlet 215 and also secured to breast cup 400. Storage compartment 210 can be opened or closed during the pumping operation. Cylinder 113 is in fluid communication with a pressure relief valve 2000 (shown in FIG. 9) that is preferably set at about 1.5 in. Hg.

Pressure relief valve 2000 has an intake 2010 and an exhaust 2050. Intake 2010 is in fluid communication with cylinder 113 and exhaust 2050 is in fluid communication with breast cup 400, by tubing 350. Pressure relief valve 2000 has a relief exhaust 2100 that is in fluid communication with intake 2010 and exhaust 2050. Relief exhaust 2100 is substantially tubular and is secured to a relief assembly 2200.

Relief assembly 2200 has a flexible insert 2210, a biasing member 2220 and a retaining member 2230. Flexible insert 2210 sealing engages with the inner surface of relief exhaust 2100 to prevent air from exiting through the relief exhaust. Insert 2210 has a securing member 2215 that mates with biasing member 2200. In this embodiment, securing member 2215 is a cross-shaped structure that is received in the inner volume of biasing member 2200. Preferably, biasing member 2220 is a spring. More preferably, biasing member 2220 is a coil spring. Retaining member 2230 is a cap-like structure having opposing retaining arms 2235 that engage with a corresponding pair of engaging protrusions 2105 positioned on the outer surface of relief exhaust 2100. Insert 2210 and spring 2220 are held in the inner volume of relief exhaust 2100 by cap 2230.

Spring 2220 has a biasing strength or resistance that is equal to the relief pressure of relief pressure valve 2000. When a positive pressure exceeds the relief pressure, which in this embodiment is preferably set at about 1.5 in. Hg, the force created on the inner surface of insert 2210 overcomes the biasing force of spring 2220 and the insert moves toward cap 2230 and outside of the inner volume of relief exhaust 2100. Air exits pressure relief valve 2000 through relief exhaust 2100 until the positive pressure in the pressure relief valve decreases below the biasing strength of spring 2220, at which time insert 2210 moves back in the inner volume of the relief exhaust, sealingly engaging the inner surfaces of the relief exhaust.

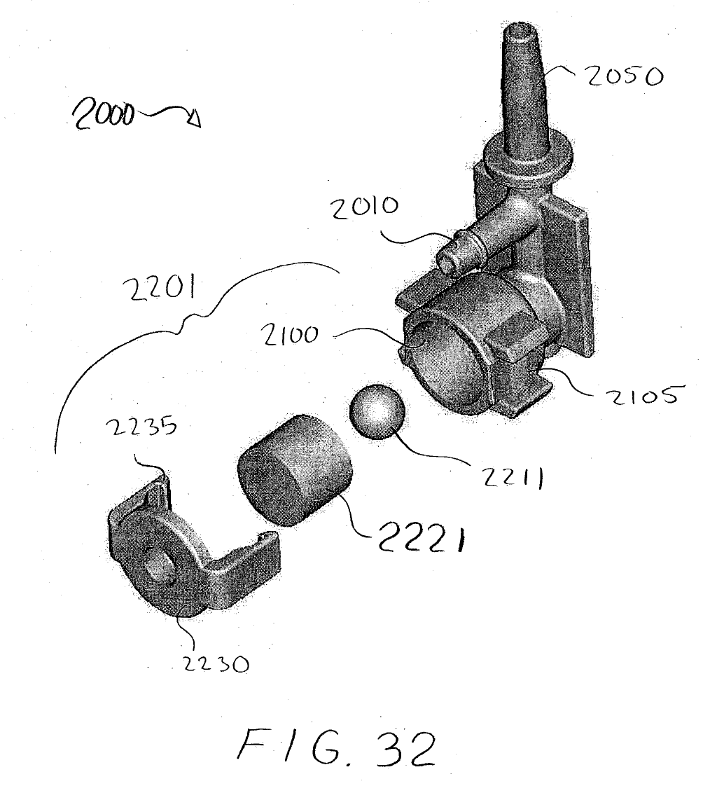

Referring also to FIG. 32, pressure relief valve 2000 is shown with a preferred relief assembly 2201 that includes an insert 2211 and a biasing member 2221. Relief assembly 2201 functions similarly to the insert 2210 and the spring 2220 of relief assembly 2200, as described above. Insert 2211 is a ball and biasing member 2221 is foam having a cylindrical shape. Relief assembly 2201 is advantageous because the ball 2211 is more easily assembled in relief exhaust 2100. Additionally, the foam cylinder 2221 is more consistent because it easily mates with the ball 2211 and provides a consistent spring actuation force. Additionally, alternative pressure relief valves can be used which are adjustable so that the “massage strength”, i.e., the amount of positive pressure on the user's breast, can be controlled.

Circuit board 120, shown in FIG. 3, allows a user to program several levels of speed and several levels of suction. In this embodiment, the speed (cycle time) ranges from about 45 cycles/minute (cpm) to about 75 cpm. The present invention provides for pre-set programming of a number of speed levels within the speed range. Preferably, the number of levels can be from about two to about eight levels. More preferably, the user can program five levels of speeds within the speed range. The present invention also envisions programming of the speed levels by the user.

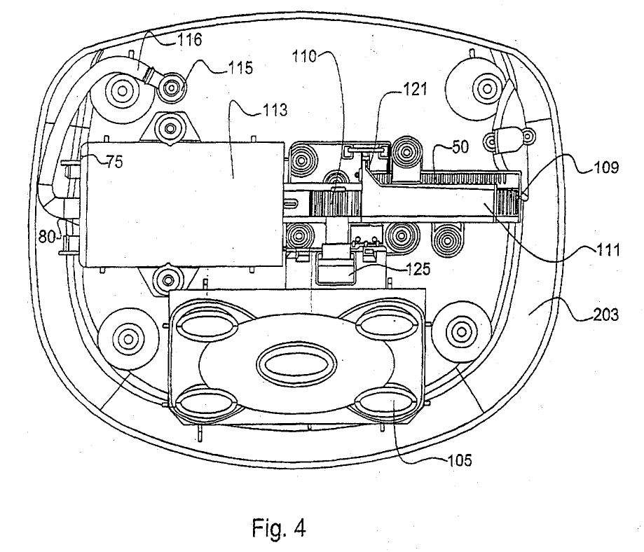

The suction range for use with a single breast cup 400 and the preferred drive system 1500 shown in FIGS. 15 through 21, is from about 3 in. Hg to about 10 in. Hg, and from about 3 in. Hg to about 8 in. Hg for two breast cups. The suction range for use with a single breast cup 400 and the gear box system shown in FIGS. 3 and 4 is from about 3 in. Hg to about 9 in. Hg, and from about 3 in. Hg to about 8 in. Hg for two breast cups. The present invention provides for pre-set programming of a number of suction levels within the suction range. Preferably, the number of levels can be from about two to about eight levels. More preferably, the user can program five levels of suction within the suction range. The present invention also envisions programming of the suction levels by the user.

Computer software can also be used to control the amount of positive and negative pressure. This allows the amounts of positive and negative pressure to be personalized for the user and also varied over the duration of the pumping process to maximize efficiency.

Breast pump 100 is preferably controlled by a software-driven circuit board 120, along with a gear motor 125, a rack and pinion set 109, 110, and a piston system 112, 113. The software and system are designed to provide maximum flexibility and to facilitate changing of the pressure curve or “wave.” This is feasible because the software controls the speed of motor 120 and the distance that piston 112 will travel in cylinder 113. The distance piston 112 travels relates to the pressure levels. By controlling speed and pressure levels with software, the pressure curve or “wave” can be controlled.

Once a determination is made that there is a specific “wave” or pressure curve that is similar to the sucking of an infant or most comfortable to the mother, then the desired wave can be obtained by changing the timing (motor speed and piston distance). Through use of software, a user has the ability to apply memory to a particular pressure curve and the variation of that pressure curve over time so as to maximize the comfort for the user.

In this embodiment, a sine wave is used for the control of breast pump 100. This is based on the assumption that the most comfortable pressure curve would be one that increases and decreases in pressure gradually, similar to a sine wave, without sharp pressure peaks and valleys providing a pinching feeling on the user. The back and forth motion of piston 112 approximates the desired sine wave. However, to avoid sharp pressure peaks, the timing of piston 112 is slowed down at these peaks, and the pressure is held constant for a duration of time at the maximum and minimum suction points on the wave. This results in a pressure curve having a steady sine wave that is more comfortable to the user.

Alternative waves can also be used for the pressure curve, if such a wave is determined to be desirable by the mother. For example, if a mother prefers a “saw tooth” pressure curve with sharp peaks, the timing of piston 112 can be changed to simply cycle back and forth, minimizing the pause when piston 112 changes direction. Also, for example, if a mother prefers a “square curve”, the timing of piston 112 can be changed to hold the piston position when the piston is ready to change direction, and then quickly ramp down and hold its position again before it ramps back up. This will create a “square curve” wave. Use of software control provides for numerous choices of waves or pressure curves. This further allows the flexibility to change or offer greater choice with one breast pump 100. In contrast, contemporary pumps have the drawback of not allowing the flexibility of changing pressure curve waves. Breast Pump 100 allows for intercycle control of the pressure and speed. In the preferred embodiment, this is done through use of a reversible, variable speed motor 125 operably connected to a linear system incorporating piston 112 and cylinder 113. Thus, contemporary devices could seemingly use a particular sinusoidal pressure curve repeatedly, while the breast pump 100 has the ability to use any type of wave and to change the wave during the cycle.

The control system and software of the present invention provide for a closed-loop control system or intercycle real-time adjustment. Thus, real-time monitoring of the control variables occurs, such as piston distance traveled and speed. As the motor and other components age and wear, the closed-loop control system accounts for such detrimental changes to provide accurate cycle time and pressure sought by the user. The real-time monitoring and control provides effectively equal speed levels for both single and double cup pumping even with the changes in torque.

Cylinder 113 has a pressure differential hole 75. Preferably, pressure differential hole 75 is located along bottom face 80 of cylinder 113. Pressure differential hole 75 is substantially smaller than exhaust hole 1013 and supply tube 116 through which the air flows for generating the positive and negative pressure. Pressure differential hole 75 provides a variance in the amount of positive pressure as compared to the amount of negative pressure. Pressure differential hole 75 is effective for the higher ranges of vacuum to provide the “lost” air at the end of the vacuum stroke. On the positive pressure stroke, a small amount of air will be released through pressure differential hole 75 but the air will be reintroduced during the negative pressure stroke when the level of pressure is higher.

Referring to FIG. 33, cylinder 113 is shown with a preferred embodiment of a pressure differential insert 76. Pressure differential hole 75 is disposed through pressure differential insert 76. Insert 76 is then connected to cylinder 113 through a cylinder hole 77 disposed through the wall of the cylinder. Insert 76 is preferably press fit into cylinder hole 77. However, alternative connection methods can also be used, such as, for example, threads or adhesive. Pressure differential insert 76 is a machined metal piece that allows for the machining of pressure differential hole 75 with a precise diameter within very small tolerances.

The use of insert 76 is advantageous over disposing pressure differential hole 75 directly through the wall of cylinder 113 because of the significant lack of precision in either molding the hole or drilling the hole through a plastic part. Additionally, pressure differential insert 76 can be selectively inserted through cylinder hole 77 so that a plurality of inserts having a plurality of differently sized pressure differential holes 75 can be used. By providing for different diameters for pressure differential hole 75, the suction levels produced by the pump can be altered.

Pressure differential hole 75 allows for reclaiming of the air during the negative pressure stroke that is lost over time during use of breast pump 100 so that the positive pressure can be accurately maintained over time. During testing of breast pump 100, unexpected and significant results occurred from the use of differently sized diameters of pressure differential hole 75. It was discovered that a pressure differential hole 75 having a diameter of between about 0.15 mm to about 0.75 mm maintained an accurate positive pressure over time while providing the desired negative pressure. The volume of cylinder 113 was 126 cm³. Preferably, pressure differential hole 75 has a diameter of between about 0.25 mm to about 0.5 mm, and most preferably the diameter is about 0.3 mm.

Referring to FIG. 10, cylinder 113 is formed as a zero-draft cylinder. The outer diameter of piston 112 creates a seal with the inner diameter d of cylinder 113 to move the volume of air inside the cylinder, creating vacuum and pressure on the breast. Breast pump 100 requires a cylinder 113 that has a consistent inner diameter d through the entire length of the cylinder to create an appropriate seal while minimizing interference or resistance to piston 112. Typical injection molded parts require a draft angle that would create a non-uniform inner diameter d of cylinder 113.

Cylinder 113 is preferably molded as a zero-draft cylinder that provides a uniform inner diameter d and more preferably, molded in a single piece. As shown in FIG. 10, cylinder 113 is a one piece, plastic injection molded part. A two-part cylinder or a machined-cylinder have drawbacks which the single piece, zero draft cylinder 113 overcomes. The two-part cylinder requires an extruded tube attached to an end cap, with the two-parts joined using a weld or using an adhesive. The machined part is typically a metal tube. One of the advantages to the zero-draft, one-piece cylinder 113 is that it is injection moldable.

Referring to FIGS. 3 through 10, button pad 105 is the user interface or control mechanism for breast pump 100. Button pad 105 has a pair of positive and negative keys for increasing or decreasing the level of suction and speed. Pad 105 further includes an on/off switch.

Due to the reciprocal back and forth motion of piston 112 in cylinder 113, breast pump 100 supplies both a positive pressure and a negative pressure to a woman's breast through a single hose or tubing 350. While this embodiment uses a piston/cylinder mechanism to create positive and negative pressure, alternative expandable volumes or pressure sources can also be used. Such alternative embodiments include a bellows mechanism or a diaphragm that would require fewer parts.

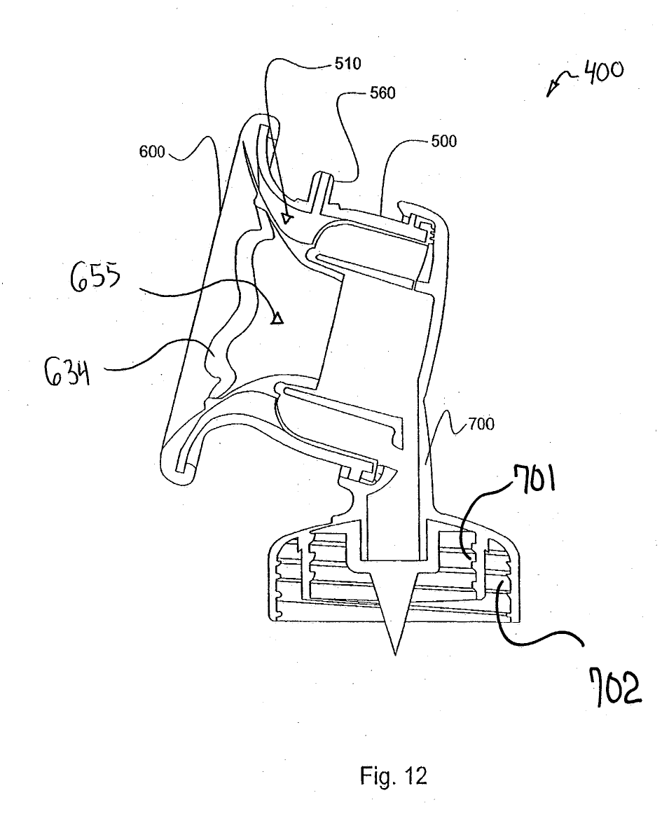

Referring to FIGS. 11 and 12, breast Cup, hood, or breast receiving member 400 of the present invention is shown. Breast cup 400 has a housing 500 having an air orifice 560, a flexible insert 600, and a holder 700. Housing 500 is a rigid structure and flexible insert 600 is a flexible structure. Housing 500 is adapted for sealing engagement with insert 600 to form a displacement volume 510 between the housing and the insert. The funnel-like shape of insert 600 provides for an inner volume 655 for receiving of the breast. Air orifice 560 is in fluid communication with displacement volume 510.

Breast pump 100 is placed in fluid communication with breast cup 400 via air tubing 350 that is connected to air orifice 560 and in fluid communication with cylinder 113. Breast pump supplies both a positive and negative pressure to breast cup 400. The positive and negative pressure created by breast pump 100 causes air to flow through air orifice 560 into and out of displacement volume 510. The positive and negative pressure supplied to breast cup 400 causes flexible insert 600 and, in particular, displacement volume 510 to expand and contract to apply reciprocating positive and negative forces on the user's breast.

Due to the negative pressure being created by evacuation of displacement volume 510 and the substantial collapsing of insert 600 upon housing 500, breast cup 400 has a maximum suction level inherently incorporated therein. Unlike contemporary devices that provide vacuum directly to the nipple from the vacuum source and are thus vulnerable to over-sucking, breast cup 400 can only provide a maximum negative pressure based upon the displacement volume 510. Once all of the air is evacuated from displacement volume 510, breast cup 400 preferably no longer increases the negative pressure or force applied to the breast. Breast pump 100 and breast cup 400 are able to apply both a positive and a negative pressure to a user's breast through a single air tubing 350, which is connected to air orifice 560.

The volume disposed in displacement volume 510 is preferably between 22 to 52 cubic centimeters, and more preferably between 32 to 42 cubic centimeters. The expandable and contractible displacement volume 510 provides an upper limit to the amount of negative pressure that can be applied to a user's breast, which can further serve as a safety feature in use of breast pump 100. Additionally, the sealing engagement of insert 600 and housing 500 provides a barrier between the user's breast and breast pump 100 to prevent any breast milk from entering air tubing 350 or the breast pump. Insert 600 can also include a massaging member 634. Massaging member 634 has a star-like shape, which provides additional massaging action to the breast. Alternative shapes can also be used for massaging member 634.

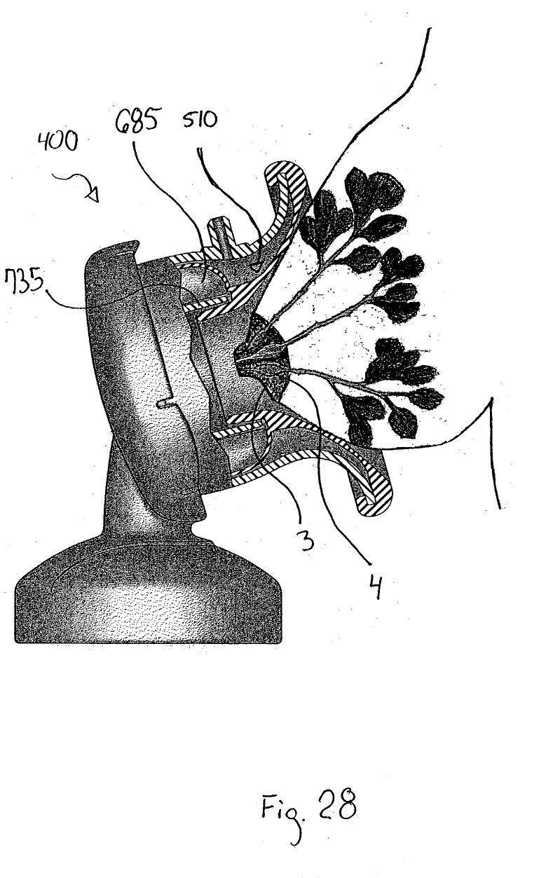

Referring to FIGS. 27 through 29, breast cup 400 is shown in partial cross-section with a breast 1. The breast 1 has a nipple 2 with an areola 3, and milk lakes or ducts 4, which are supplied by milk glands 5. Breast cup 400 has bladders 685 on insert 600 and tubular member 735 on holder 700. Bladders 685 partially define displacement volume 510. When air is evacuated from the bladders 685 and the displacement volume 510 such that insert 600 is pulled toward and against housing 500, then the negative pressure, vacuum or negative force is applied to breast 1.

Tubular member 735 is disposed substantially adjacent to bladders 685 and extends partially through insert 600. Tubular member 735 is a rigid barrier between the breast 1 and bladders 685 to prevent the breast from making contact with and impinging the bladders, which would reduce the amount of their inflation and deflation, and thus reduce the reciprocating pressure applied to the breast.

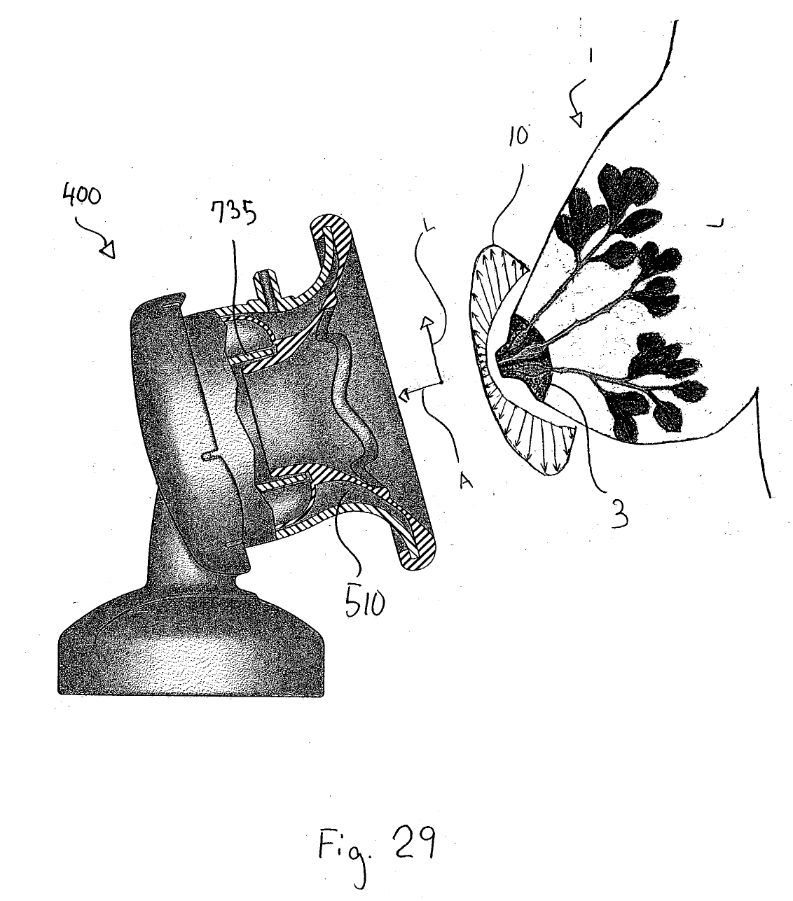

Positioning of the breast cup 400 on the breast 1, results in the nipple 2, areola 3 and milk ducts 4 being substantially surrounded by displacement volume 510. Nipple 2 being substantially surrounded by the displacement volume and the use of tubular member 735 to create a rigid barrier in front of areola 3 and adjacent to bladders 685, results in a negative pressure gradient, vacuum or negative force 10 being applied to nipple 2 upon evacuation of the air in displacement volume 510 during the negative pressure stroke or cycle, as represented in FIG. 29. The negative pressure gradient or force 10 has a lateral component or direction L that is greater than an axial component or direction A. The negative pressure gradient or force 10 and the larger lateral component L causes the nipple 2 to be pulled or sucked laterally more than axially, which has been shown to be significantly more efficient at causing expression of breast milk from the milk ducts 4. The negative pressure gradient or force 10 has also been shown to be more comfortable for the user and more like the sucking of a baby during breast-feeding, due in part to the widening of the nipple 2 as opposed to axially elongating or distending the nipple along axial direction A.

Displacement volume 510 extends almost to the leading edge of housing 500 where the housing is secured to insert 600, which assists in creating the negative pressure gradient or force 10 during the negative pressure stroke or cycle that causes lateral sucking and lateral movement of the nipple 2 along the lateral component L. As shown in FIG. 29, the negative pressure gradient, vacuum or force 10 extends beyond the outer circumference of the areola 3 and is substantially laterally applied thereto during the negative pressure stroke or cycle, which further assists in creating a force on the nipple 2 with a greater lateral component L than axial component A and thus a widening of the nipple.

The positioning of tubular member 735 helps reduce the negative pressure gradient or force 10 axially from or in front of, the nipple 2 during the negative pressure stroke or cycle, which reduces discomfort associated with axial distention of the nipple. Tubular member 735 has an opening (not shown) formed along the tubular member wall. For softer breasts 1, which are pulled into the tubular member 735 during the negative pressure stroke, the opening allows application of the negative pressure or vacuum to the distal end of the nipple 2.

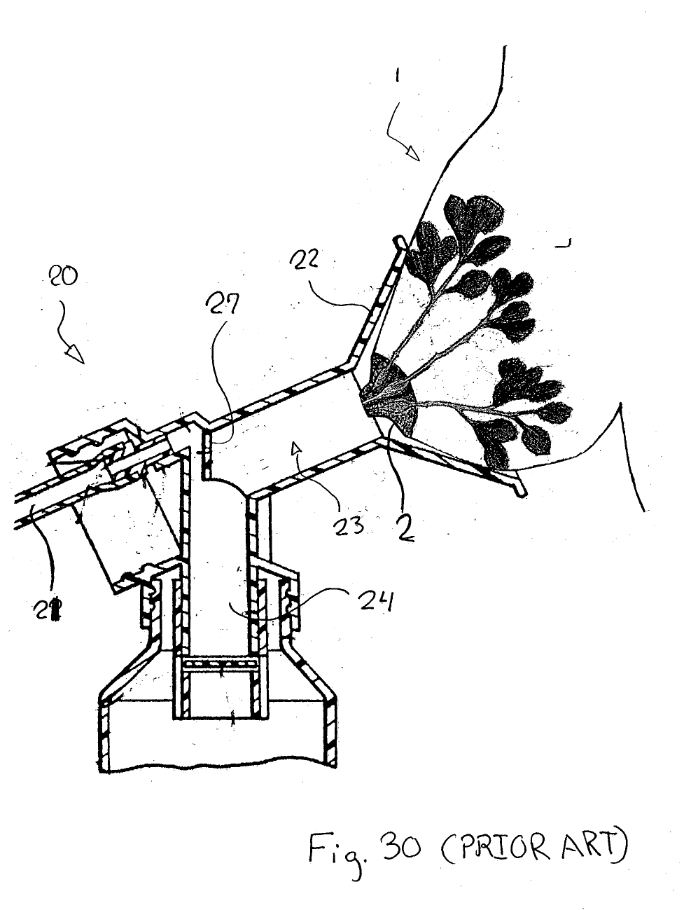

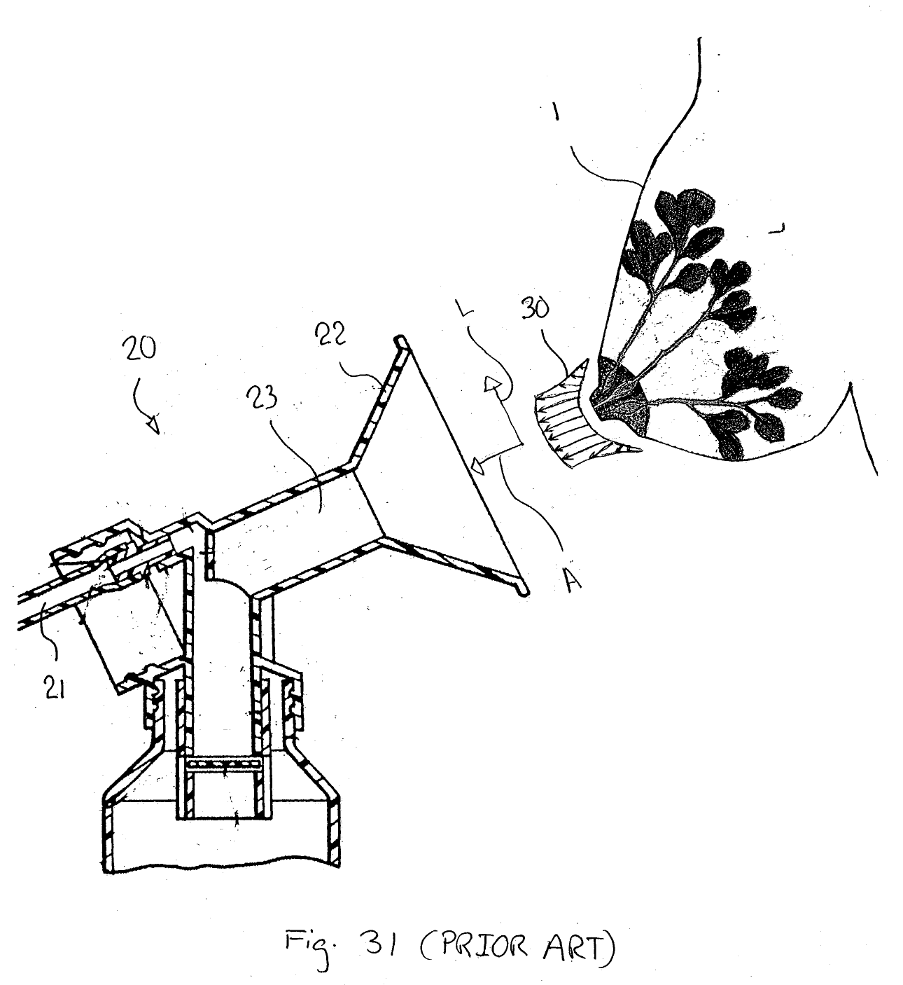

Referring to FIGS. 30 through 31, a contemporary breast cup 20 is shown which is connected to a vacuum source through a vacuum line 21. The contemporary breast cup 20 has a hood 22 that can engage the breast 1 and a cylindrical extension 23 attached to the hood. The cylindrical extension 23 is in fluid communication with the vacuum line 21 and a collection member 24. The vacuum or negative pressure is supplied from the vacuum line through the cylindrical extension 23 and to the areola 2. A separation wall 27 seemingly prevents the breast milk from entering the vacuum line 21. The evacuation of the air in cylindrical extension 23 creates a negative pressure gradient or force 30 during the negative pressure stroke, as represented in FIG. 31.

The negative pressure gradient or force 30 has a greater axial component A than lateral component L during the negative pressure stroke, causing the nipple 2 to be pulled or sucked axially more than laterally, which has been shown to be significantly less efficient at causing expression of breast milk from the milk ducts. The negative pressure gradient or force 30 having a greater axial component A than lateral component L during the negative pressure stroke, has also been shown to be uncomfortable for the user. The vacuum or negative pressure is supplied axially from or in front of, nipple 2 during the negative pressure stroke or cycle,,which causes discomfort associated with axial elongation and distention of the nipple.

While breast cup 400 uses a flexible insert 600 partially defining a displacement volume 510 that applies the negative pressure gradient, vacuum or force 10 to the nipple 2 during the negative pressure stroke or cycle, the present invention contemplates the use of other designs and arrangements that create the negative pressure gradient, vacuum or force 10. Alternative designs for breast cup 400 that cause greater widening of the nipple along the lateral component L as opposed to elongation or distention of the nipple along the axial component A during the negative pressure stroke or cycle are contemplated by the present invention. Also, alternative designs for breast cup 400 that apply a negative force to nipple 2 during the negative pressure stroke having an average lateral component L that is greater than the average axial component A are contemplated by the present invention. Further, alternative designs for breast cup 400 that apply a negative pressure gradient or vacuum to nipple 2 during the negative pressure stroke having an average lateral component L that is greater than the average axial component A are contemplated by the present invention.

While the preferred embodiment describes the use of a motorized pump 100 that supplies the pressure to breast cup 400, the present invention contemplates the use of manual pumps for use with breast cup 400, including pumping mechanisms that are affixed to breast cup 400. Additionally, the present invention contemplates other barrier structures, designs or methods which reduce the negative pressure, vacuum or negative force applied to the distal end or front of nipple 2, and/or reduce the axial component A of the negative pressure, vacuum or negative force applied to the nipple 2, as compared to the lateral component L.

The present invention contemplates the use of a valve or other known release mechanism (not shown) in fluid communication with displacement volume 510 so that a user could alternatively selectively control the amount of positive or negative pressure at the breast cup 400 rather than only at the breast pump 100. The valve or release mechanism on the breast cup 400 could also be a quick release mechanism as a safety feature in the event of discomfort to the user. The valve or release mechanism could also be used to selectively allow only positive or negative pressure to be generated at the breast cup 400.

The modularity of breast cup 400 through use of three separate pieces that can be easily assembled, i.e., housing 500, insert 600 and holder 700, allows the present invention to include a kit to accommodate breasts of varying sizes and shapes. The kit can include a plurality of differently sized housings 500 and inserts 600, as well as differently shaped housings 500 and inserts 600, to accommodate different sized breasts and different shaped breasts. The plurality of different housings 500 and inserts 600 can all be assembled to holder 700 and can be connected to breast pump 100. An example of the variation in sizes of housings 500 and inserts 600 includes the inner and outer diameters throughout the housings and inserts, as well as the length of the housings and inserts. An example of the variation in shapes of housings 500 and inserts 600 includes varying the taper angle, as well as changing the circular shape of the leading edge of the housing and insert. Additionally, the modularity and interchangeability of the present invention allows for the use of different shaped or sized massaging members or projections 634 on different inserts 600.

The present invention also contemplates a kit containing a plurality of differently sized or shaped inserts 600 that can all be assembled to housing 500 and holder 700, to form a plurality of different breast cups 400 for use with breast pump 100. The plurality of differently sized inserts 600 can be used to accommodate different sized breasts and also to change the displacement volume 510. The plurality of differently shaped inserts 600 can be used to accommodate differently shaped breasts, as well as to provide different massaging effects to the breasts, such as, for example, different massaging members 634 formed on the insert. Examples of some alternative inserts 600 are described more fully in copending U.S. application Ser. No. 10/331,183, filed Dec. 27, 2002, the disclosure of which has been incorporated by reference herein in its entirety.

While the preferred embodiment of the breast pump system uses breast cup 400 having a displacement volume 510 in fluid isolation from the user's breast, alternative breast cups can also be used with breast pump 100. The unique features of the breast pump system of the present invention can be used with other types of breast cups, such as, for example, the control system of the present invention or the rack and pinion driving mechanism.

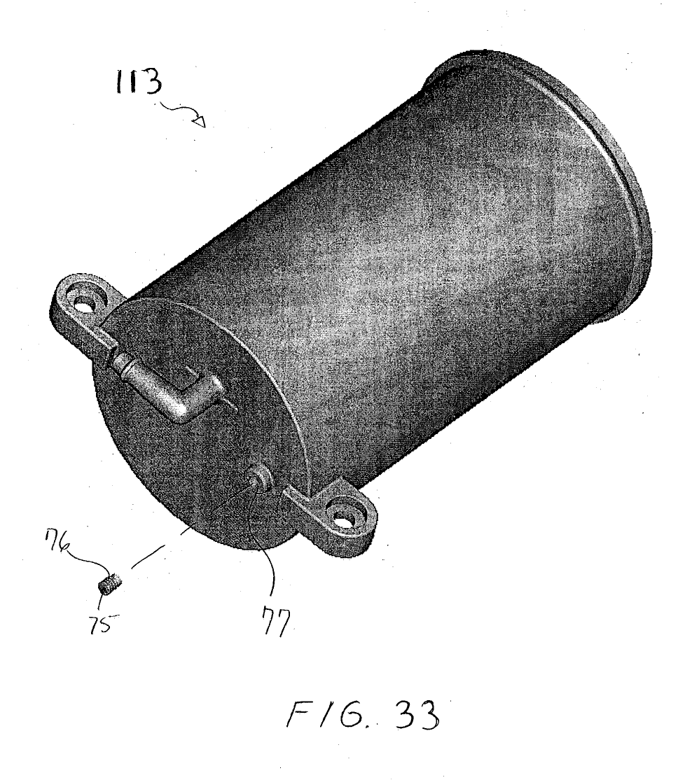

Referring to FIG. 34, an alternative embodiment of the breast cup of the present invention is shown and generally represented by reference numeral 5400. Breast cup 5400 is usable with insert 600. Breast cup 5400 has a funnel shaped housing 5500 that is connected to a cylindrically-shaped holder 5700. Holder 5700 has a handle 5725, a pressure orifice 5750, and a pressure adjuster 5775. Handle 5725 is ergonomically contoured and has a wave-like shape 5730 that provides for different holding angles. Handle 5725 is disposed along holder 5700 on the opposing side from funnel 5500. Handle 5725 is preferably made of, or covered by, a material that facilitates gripping. Handle 5725 can include various textures, projections and/or embossments to sooth the users hand during the pumping process.

Pressure orifice 5750 can be attached to tubing 350 to place breast cup 5400 in fluid communication with breast pump 100. Pressure adjuster 5775 is in fluid communication with pressure orifice 5750 and allows a user to adjust the pressure at the breast cup 5400 without having to make an adjustment at the breast pump 100. In this embodiment, pressure adjuster 5775 is a dial but alternative actuators can also be used.

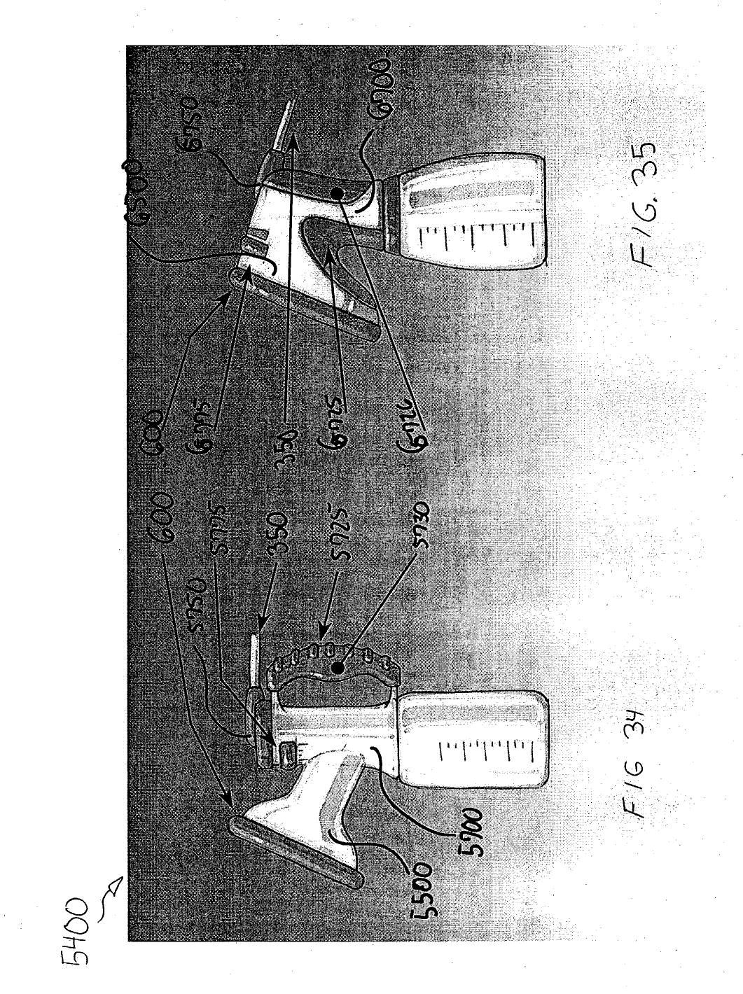

Referring to FIG. 35, another alternative embodiment of the breast cup of the present invention is shown and generally represented by reference numeral 6400. Breast cup 6400 is usable with insert 600. Breast cup 6400 has a funnel 6500 that is connected to a holder 6700. Holder 6700 has handle portions 6725, 6726, a pressure orifice 6750, and a pressure adjuster 6775. Handle portions 6725, 6726 are disposed on opposing sides of holder 6700 and facilitate grasping of the holder. Handle portions 6725, 6726 are preferably made of, or covered by, a material that facilitates gripping. Handle portions 6725, 6726 can include various textures, projections and/or embossments to sooth the users hand during the pumping process.

Referring back to FIG. 12, holder 700 of breast cup 400 provides a first set of threads 701 and a second set of threads 702. First and second threads 701, 702 have different diameters and are sized to fit the two standard sized bottles or holders that are used with infant feeding and breast pumping, i.e., reusable containers and disposable containers. The first and second threads 701, 702 have the same pitch and are concentrically aligned. During the molding process, this allows the steel mold core to be unscrewed from holder 700.

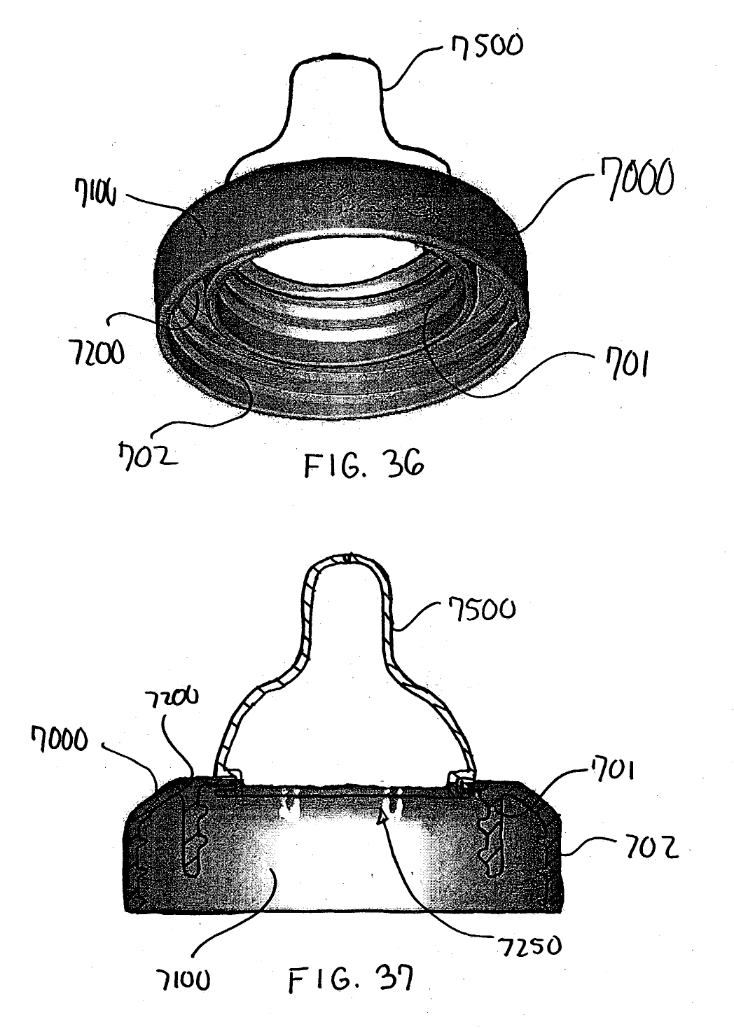

While the embodiment illustrated shows the dual threads, i.e., first and second threads 701, 702 on breast cup 400, the present invention contemplates the use of the dual threads on other infant care products that require the use of a holder or bottle, such as, for example, a nipple ring or a cap. Referring to FIGS. 36 and 37, a nipple ring is shown and generally represented by reference numeral 7000. Nipple ring 7000 has a circumferential wall 7100 with an inwardly extending flange 7200 defining an opening 7250. Nipple ring 7000 has the dual threads described above, i.e., a first set of threads 701 and a second set of threads 702. The nipple ring 7000 provides for engagement of nipple 7500 with either reusable containers by way of first threads 701 or disposable containers by way of second threads 702. Preferably, first threads 701 downwardly extend from flange 7200 and second threads 702 ate formed along circumferential wall 7100.

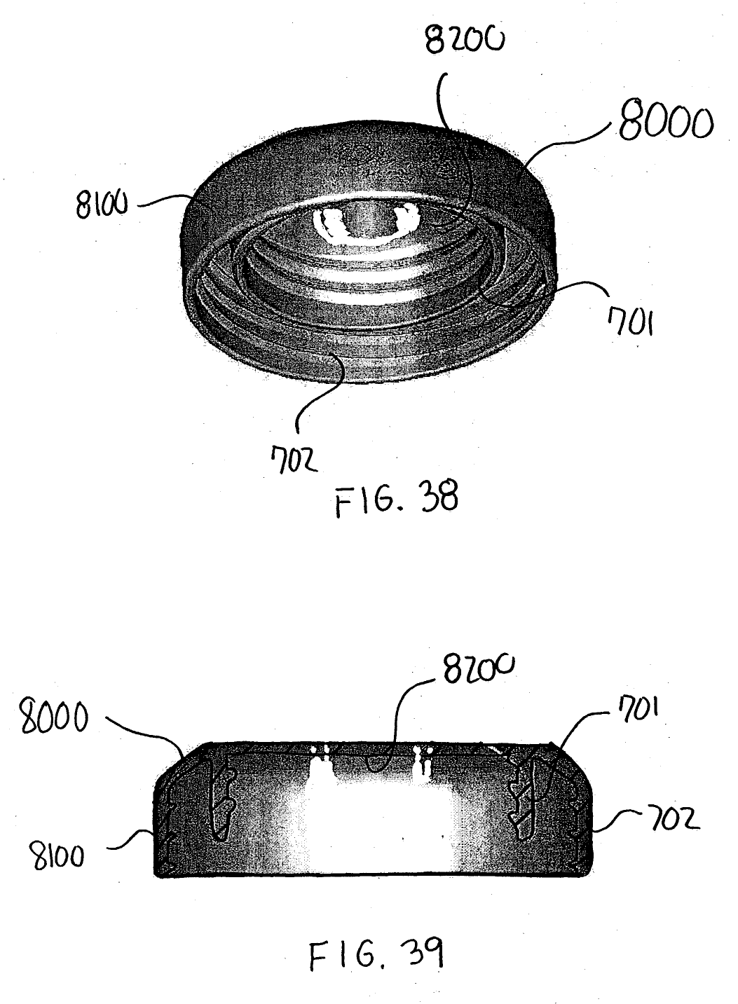

Referring to FIGS. 38 and 39, a cap is shown and generally represented by reference numeral 8000. Cap 8000 has a circumferential wall 8100 connected to a top wall 8200. Cap 8000 also has the dual threads described above, i.e., a first set of threads 701 and a second set of threads 702. The cap 8000 provides sealing of either reusable containers by way of first threads 701 or disposable containers by way of second threads 702. Preferably, first threads 701 downwardly extend from top wall 8200 and second threads 702 are formed along circumferential wall 8100.

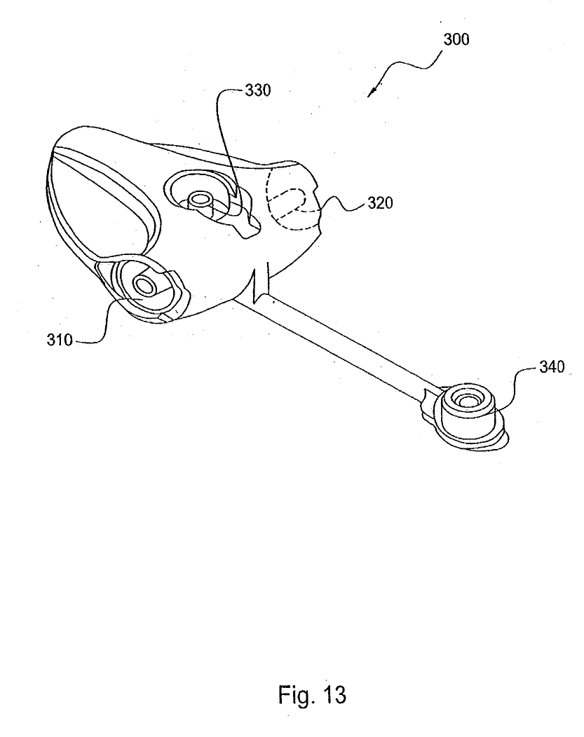

Referring to FIG. 13, T-connector 300 is a triangular shaped valve that allows a user to utilize either a single breast cup 400 or two breast cups through use of a first orifice 310 and a second orifice 320. Breast pump 100 is connected to t-connector 300 through air tubing 350 at inlet 330. The single split valve configuration of t-connector 300 minimizes the amount of tubing 350 necessary for double pumping. T-connector 300 has a plug 340 for closing off either of first or second orifices 310, 320 if single pumping is desired. Preferably, plug 340 is tethered to an outer surface of t-connector 300 to facilitate engagement with first or second orifices 310, 320.

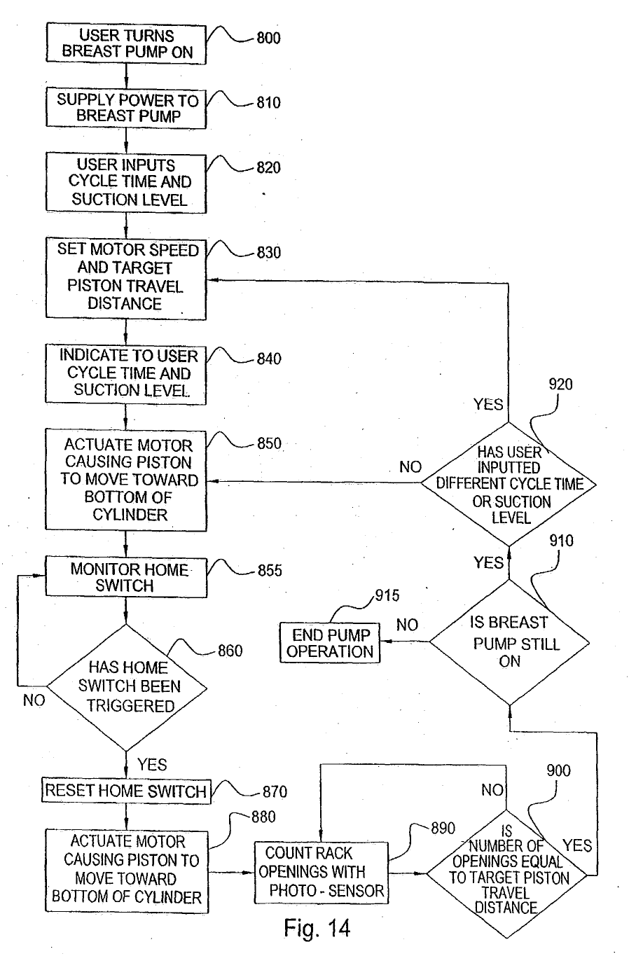

Referring to FIG. 14, a method of expressing breast milk according to the breast pump system of the present invention, is shown. The user commences the breast pumping operation by turning breast pump 100 “on,” as in step 800. This causes power to be supplied to breast pump 100 (step 810). The user then inputs the cycle time and suction level that is desired, as in step 820. In the preferred embodiment, the user has five cycle times and suction levels from which to choose. The cycle time and suction level is inputted by use of button pad 105.

In step 830, PC board 120 sets the motor'speed and target piston travel distance according to the user's inputted levels for cycle time and suction. The cycle time and suction level are then displayed to the user, as in step 840. In this embodiment, the cycle time and suction level are indicated by lights 225 with the number of illuminated lights corresponding to the level. In step 850, motor 125 is actuated causing piston 112 to move toward bottom 175 of cylinder 113. This creates a positive pressure that is supplied to breast cup 400 by air tubing 350.

In step 855, the PC Board monitors the home switch to determine whether it has been triggered by contact with piston 112. In step 860, it is determined whether the home switch has been triggered. If the home switch has been triggered then it is reset as in step 870. In step 880, motor 125 is then reversed causing piston 112 to move toward top 180 of cylinder 113. This creates a negative pressure that is supplied to breast cup 400 by air tubing 350. One of the advantages of the breast pump system of the present invention is that is supplies both a positive pressure and a negative pressure through the same air tubing 350. This reduces cleaning and simplifies the operation for a user.

To provide the proper amount of suction as inputted by the user, photo-sensors 121 count the number of rack openings 50, as in step 890. In step 900, PC board 120 determines if the number of rack openings 50 that have been counted is the equivalent of the target piston travel distance as inputted by the user. In step 910, it is determined whether breast pump 100 is still “on.” If breast pump 100 has been shut off then the pumping operation ends, as in step 915.

In step 920, it is determined whether the user has inputted a new cycle time or suction level. If a new cycle time or suction level has been inputted, then PC Board 120 sets the motor speed and target piston travel distance according to the user's inputted levels for cycle time and suction, reverting back to step 830 and repeating the above described steps. If the user has not inputted a new cycle time or suction level then the motor is again reversed causing piston 112 to move toward bottom 175 of cylinder 113. This creates a positive pressure that is supplied to breast cup 400 by air tubing 350. The process continues with breast pump 100 supplying positive pressure and then negative pressure to breast cup 400 until the breast pump is shut off (step 910).

The breast pump system of the present invention includes a number of components and can be used in remote locations, such as when a user is traveling. The various components can be disposed within a bag system for ease of use. An example of such a bag system, as well as the components of such a system, is disclosed in the co-pending and commonly owned U.S. application Ser. No. 10/331,130, filed Dec. 27, 2002, the disclosure of which is incorporated herein by reference.

The present invention having been thus described with particular reference to the preferred forms thereof, it will be obvious that various changes and modifications may be made therein without departing from the spirit and scope of the present invention as defined in the appended claims.

What is claimed is:

1. A breast cup in fluid communication with a pressure source and adapted for expressing breast milk from a breast having a nipple, the breast cup comprising:
a hood for receiving the breast and in fluid communication with the pressure source, wherein said hood creates a negative force on the nipple during a negative pressure stroke, said negative force having a lateral component and an axial component, and wherein said lateral component is greater than said axial component.

2. The breast cup of claim 1, further comprising a barrier member operably connected to said hood, wherein said barrier member reduces said axial component of said negative force during said negative pressure stroke.

3. The breast cup of claim 1, wherein said hood comprises a housing, a flexible insert sealingly secured to said housing, and a displacement volume formed between said housing and said flexible insert, wherein said displacement volume is in fluid communication with the pressure source.

4. The breast cup of claim 3, wherein said displacement volume substantially surrounds the nipple when the breast is received in said hood.

5. The breast cup of claim 3, wherein said flexible insert comprises a bladder in fluid communication with the pressure source, said displacement volume being defined at least partially by said bladder, and wherein said bladder and said displacement volume contract to form said negative force on the nipple during said negative pressure stroke.

6. The breast cup of claim 5, further comprising a barrier member disposed substantially adjacent to said bladder, said barrier member preventing the breast from contacting said bladder.

7. The breast cup of claim 6,, wherein said flexible insert defines an inner volume for receiving the breast, and wherein said barrier member has a cylindrical shape and is disposed in said inner volume.

8. The breast cup of claim 3, wherein said flexible insert has a funnel shape with a massaging projection formed thereon.

9. The breast cup of claim 8, wherein said massaging projection has a star-like shape.

10. A breast cup connected to a vacuum source and adapted for expressing breast milk from a breast having a nipple, the breast cup comprising:
a breast receiving member in fluid communication with the vacuum source, wherein said breast receiving member applies a negative pressure to the nipple during a negative pressure stroke causing the nipple to widen along a lateral direction.

11. The breast cup of claim 10, wherein said negative pressure causes the nipple to widen along a lateral direction more than the nipple elongates along an axial direction.

12. The breast cup of claim 10, wherein said negative pressure has an average lateral component and an average axial component, and wherein during said negative pressure stroke said average lateral component is greater than said average axial component.

13. The breast cup of claim 10, further comprising a barrier member operably connected to said breast receiving member, wherein said barrier member reduces elongation of the nipple along said axial direction during said negative pressure stroke.

14. The breast cup of claim 10, wherein said breast receiving member comprises a housing, a flexible insert sealingly secured to said housing, and a displacement volume formed between said housing and said-flexible insert, wherein said displacement volume is in fluid communication with said vacuum source.

15. The breast cup of claim 14, wherein said displacement volume substantially surrounds the nipple when the breast is received in said breast receiving member.

16. The breast cup of claim 14, wherein said flexible insert comprises a bladder in fluid communication with the vacuum source, said displacement volume being defined at least partially by said bladder, and wherein said bladder and said displacement volume contract during said negative pressure stroke to form a negative force on the nipple.

17. The breast cup of claim 16, further comprising a barrier member disposed substantially adjacent to said bladder, said barrier member preventing the breast from contacting said bladder.

18. The breast cup of claim 17, wherein said flexible insert defines an inner volume for receiving the breast, and wherein said barrier member has a cylindrical shape and is disposed in said inner volume.

19. The breast cup of claim 14, wherein said flexible insert has a funnel shape with a massaging projection formed therein.

20. A breast pump system for expressing breast milk from a breast having a nipple, the system comprising:
a pressure source; and
a breast cup for receiving the breast and in fluid communication with said pressure source, wherein said breast cup creates a negative force on the nipple during a negative pressure stroke, said negative force having a lateral component and an axial component, and wherein said lateral component is greater than said axial component.

21. The breast pump system of claim 20, further comprising a barrier member operably connected to said breast cup, wherein said barrier member reduces said axial component of said negative force during said negative pressure stroke.

22. The breast pump system of claim 20, wherein said breast cup comprises a housing, a flexible insert sealingly secured to said housing, and a displacement volume formed between said housing and said flexible insert, wherein said displacement volume is in fluid communication with said pressure source.

23. The breast pump system of claim 22, wherein said displacement volume substantially surrounds the nipple when the breast is received in said breast cup.

24. The breast pump system of claim 22, wherein said flexible insert comprises a bladder in fluid communication with the pressure source, said displacement volume being defined at least partially by said bladder, and wherein said bladder and said displacement volume contract to form said negative force on the nipple during said negative pressure stroke.

25. The breast pump system of claim 24, further comprising a barrier member disposed substantially adjacent to said bladder, said barrier member preventing the breast from contacting said bladder.

26. The breast pump system of claim 22, wherein said flexible insert has a funnel shape with a massaging projection formed therein.

27. A breast pump system for expressing breast milk from a breast having a nipple, the system comprising:
a vacuum source; and
a breast receiving member in fluid communication with said vacuum source, wherein said breast receiving member applies a negative pressure to the nipple during a negative pressure stroke causing the nipple to widen along a lateral direction.

28. The breast pump system of claim 27, wherein said negative pressure causes the nipple to widen along a lateral direction more than the nipple elongates along an axial direction.

29. The breast pump system of claim 27, wherein said negative pressure has an average lateral component and an average axial component, and wherein said average lateral component is greater than said average axial component during said negative pressure stroke.

30. The breast pump system of claim 27, further comprising a barrier member operably connected to said breast receiving member, wherein said barrier member reduces elongation of the nipple along said axial direction during said negative pressure stroke.

31. The breast pump system of claim 27, wherein said breast receiving member comprises a housing, a flexible insert sealingly secured to said housing, a displacement volume formed between said housing and said flexible insert, and an air orifice in fluid communication with said displacement volume and the vacuum source.

32. The breast pump system of claim 31, wherein said displacement volume substantially surrounds the nipple when the breast is received in said breast receiving member.

33. The breast pump system of claim 31, wherein said flexible insert comprises a bladder in fluid communication with said vacuum source, said displacement volume being defined at least partially by said bladder, and wherein said bladder and said displacement volume contract during said negative pressure stroke to form a negative force on the nipple.

34. The breast pump system of claim 33, further comprising a barrier member disposed substantially adjacent to said bladder, said barrier member preventing the breast from contacting said bladder.

35. The breast pump system of claim 34, wherein said flexible insert defines an inner volume for receiving the breast, and wherein said barrier member has a cylindrical shape and is disposed in said inner volume.

36. The breast pump system of claim 31, wherein said flexible insert has a funnel shape with a massaging projection formed thereon.

37. The breast pump system of claim 27, wherein said vacuum source is a piston movably disposed in a cylinder.

38. The breast pump system of claim 37, further comprising a reversible motor operably connected to said piston.

39. The breast pump system of claim 38, further comprising a rack having first teeth and a gear having second teeth, wherein said rack is connected to said piston, said gear is operably connected to said reversible motor, and said first teeth engage with said second teeth to reciprocally move said piston in said cylinder.

40. The breast pump system of claim 37, wherein said cylinder has a first diameter and an air hole, said air hole has a second diameter and is in fluid communication with atmosphere, and said first diameter is significantly larger than said second diameter.

41. The breast pump system of claim 38, further comprising a controller operably connected to said reversible motor, wherein said controller determines a distance that said piston has traveled relative to said cylinder, and wherein said controller reverses said motor based upon said distance.

42. The breast pump system of claim 38, further comprising a controller operably connected to said reversible motor, wherein said motor is variable speed and said controller adjusts said speed based upon a desired cycle time for applying said negative pressure to the breast.

43. A breast pump kit for use with a pressure source for expressing breast milk from a breast, the kit comprising:
a holder; and
a plurality of hoods for receiving the breast, each of said plurality of hoods being selectively engageable to said holder and the pressure source for expressing the breast milk from the breast, wherein at least one of said plurality of hoods has a different size or a different shape than another of said plurality of hoods.

44. The breast pump kit of claim 43, wherein each of said plurality of hoods comprises a housing, a flexible insert sealingly secured to said housing, and a displacement volume formed between said housing and said flexible insert and in fluid communication with the pressure source.

45. The breast pump kit of claim 44, wherein said housing and said flexible insert of said at least one of said plurality of hoods has a different size or a different shape than said housing and said flexible insert of said another of said plurality of hoods.

46. The breast pump kit of claim 44, wherein said housing of said at least one of said plurality of hoods has a different size or a different shape than said housing of said another of said plurality of hoods.

47. The breast pump kit of claim 44, wherein said flexible insert of said at least one of said plurality of hoods has a different size or a different shape than said flexible insert of said another of said plurality of hoods.

48. The breast pump kit of claim 43, further comprising a container, wherein said holder is selectively engageable with said container.

49. The breast pump kit of claim 48, wherein said holder has a plurality of engagement structures for selectively engaging to a plurality of different sized containers.

50. The breast pump kit of claim 47, wherein said flexible insert of said at least one of said plurality of hoods has a first massaging projection, wherein said flexible insert of said another of said plurality of hoods has a second massaging projection, and wherein said first and second massaging projections have a different size or a different shape.

51. A breast pump system for expressing breast milk from a breast, the system comprising:
a pump generating pressure; and
a plurality of hoods for receiving the breast, each of said plurality of hoods being selectively fluidly connectable to said pump for expressing the breast milk from the breast, wherein at least one of said plurality of hoods has a different size or a different shape than another of said plurality of hoods.

52. The system of claim 51, further comprising a holder selectively engageable with each of said plurality of hoods.

53. The system of claim 51, wherein each of said plurality of hoods comprises a housing, a flexible insert sealingly secured to said housing, and a displacement volume formed between said housing and said flexible insert and in fluid communication with said pump.

54. The system of claim 53, wherein said housing and said flexible insert of said at least one of said plurality of hoods has a different size or a different shape than said housing and said flexible insert of said another of said plurality of hoods.

55. The system of claim 53, wherein said housing of said at least one of said plurality of hoods has a different size or a different shape than said housing of said another of said plurality of hoods.

56. The system of claim 53, wherein said flexible insert of said at least one of said plurality of hoods has a different size or a different shape than said flexible insert of said another of said plurality of hoods.

57. The system of claim 52, further comprising a container, wherein said holder is selectively engageable with said container.

58. The system of claim 57, wherein said holder has a plurality of engagement structures for selectively engaging to a plurality of different sized containers.

59. The system of claim 56, wherein said flexible insert of said at least one of said plurality of hoods has a first massaging projection, wherein said flexible insert of said another of said plurality of hoods has a second massaging projection, and wherein said first and second massaging projections have a different size or a different shape.

60. A breast pump for expressing breast milk from a breast, the pump comprising:
a pressure source having a movable structure for generating pressure during a pressure stroke, said movable structure having a variable pressure volume or variable cycle time; and
a controller operably connected to said pressure source, wherein said controller regulates said pressure volume based upon a distance traveled by said movable structure or regulates said variable cycle time based upon a speed of said movable structure, and wherein said controller provides substantially real-time monitoring of said distance traveled or said speed.

61. The pump of claim 60, wherein said controller can regulate said pressure cycle based upon a non-sinusoidal wave signal of said pressure versus said variable cycle time.

62. A breast pump system for expressing breast milk from a breast comprising:
a pressure source having an evacuation volume for generating a pressure and an air hole; and
a breast cup for receiving the breast and in fluid communication with said pressure source for applying said pressure to the breast, wherein said air hole has a diameter, said air hole being in fluid communication with atmosphere and said evacuation volume, and wherein said diameter is between about 0.15 mm to 0.75 mm.

63. The system of claim 62, wherein said diameter is between about 0.25 mm to about 0.5 mm.

64. The system of claim 62, wherein said diameter is about 0.3 mm.

65. A method of expressing breast milk from a breast comprising:
applying a negative pressure on the breast from a pressure source during a vacuum stroke;
applying a positive pressure on the breast from said pressure source during a massage stroke; and
providing air from atmosphere to said pressure source during said vacuum stroke.

66. The method of claim 65, wherein the step of providing air from atmosphere to said pressure source during said vacuum stroke comprises providing an air hole in fluid communication with said pressure source and the atmosphere, said air hole having a diameter between about 0.15 mm to 0.75 mm.

67. A method of expressing breast milk from a breast having a nipple comprising:
applying a negative pressure on at least a portion of the nipple causing the nipple to widen along a lateral direction.

68. The method of claim 67, wherein said negative pressure causes the nipple to widen along said lateral direction more than the nipple elongates along an axial direction.

69. The method of claim 67, wherein said negative pressure has an average lateral component and an average axial component during a negative pressure stroke, and wherein said average lateral component is greater than said average axial component.

70. A method of expressing breast milk from a breast comprising:
applying a pressure to the breast; and
performing substantially real-time monitoring and controlling of said pressure with a controller.

71. The method of claim 70, wherein said pressure is controlled in part based upon a variable pressure volume of a movable structure or a variable cycle time of said movable structure, wherein said controller regulates said pressure volume based upon a distance traveled by said movable structure or regulates said variable cycle time based upon a speed of said movable structure, and wherein said controller provides substantially real-time monitoring of said distance traveled or said speed.

72. The method of claim 70, wherein said controller can regulate said pressure based upon a non-sinusoidal wave signal of said pressure versus a cycle time.

73. A pump for providing pressure comprising:
a housing defining a volume and having a pressure exhaust;
an actuator operably connected to said housing for producing the pressure in said volume; and
an insert connected to said housing, said insert having a hole disposed therethrough, said hole providing fluid communication between said volume and atmosphere.

74. The pump of claim 73, wherein said housing is a first material, and said insert is a second material.

75. The pump of claim 74, wherein said housing is plastic, and wherein said insert is metal.

76. The pump of claim 73, wherein said housing is a cylinder, and wherein said actuator is a piston.

77. The pump of claim 76, wherein said cylinder has an orifice, and wherein said insert is disposed in said orifice.

78. The pump of claim 77, wherein said insert is press fit in said orifice.

79. The pump of claim 76, wherein said insert is a plurality of inserts, and wherein each of said plurality of inserts is selectively engageable with said cylinder.

80. The pump of claim 77, wherein said insert is a plurality of inserts, and wherein each of said plurality of inserts is selectively engageable with said orifice.

81. A breast cup for placing a breast in fluid, communication with a first container and a second container, the first and second containers having openings with different diameters, the breast cup comprising:
a funnel for receiving the breast;
a housing connected to the funnel and having a base, said base having a circumferential wall, a flange extending inwardly from said circumferential wall to define an opening, first threads, and second threads,
wherein said first threads have a first diameter and a first pitch, said first diameter and said first pitch allowing for selective engagement with the first container, wherein said second threads have a second diameter and a second pitch, said second diameter and said second pitch allowing for selective engagement with the second container, and wherein said first threads and said second threads are concentrically disposed along said base.

82. The breast cup of claim 81, wherein said first pitch is equal to said second pitch.

83. The breast cup of claim 81, wherein said first threads extend from said flange, and wherein said second threads are disposed on said circumferential wall.

84. The breast cup of claim 81, wherein said funnel is selectively removable from said housing.

85. A nipple ring for engaging a nipple with a first container and a second container, the first and second containers having openings with different diameters, the nipple ring comprising:
a body having a circumferential wall, a flange extending inwardly from said circumferential wall to define an opening, first threads, and second threads,
wherein said first threads have a first diameter and a first pitch, said first diameter and said first pitch allowing for selective engagement with the first container, wherein said second threads have a second diameter and a second pitch, said second diameter and said second pitch allowing for selective engagement with the second container, and wherein said first threads and said second threads are concentrically disposed along said body.

86. The nipple ring of claim 85, wherein said first pitch is equal to said second pitch.

87. The nipple ring of claim 85, wherein said first threads extend from said flange, and wherein said second threads are disposed on said circumferential wall.

88. A cap for engaging with a first container and a second container, the first and second containers having openings with different diameters, the cap comprising:
a body having a circumferential wall, a top wall connected to said circumferential wall, first threads, and second threads,
wherein said first threads have a first diameter and a first pitch, said first diameter and said first pitch allowing for selective engagement with the first container, wherein said second threads have a second diameter and a second pitch, said second diameter and said second pitch allowing for selective engagement with the second container, and wherein said first threads and said second threads are concentrically disposed along said body.

89. The cap of claim 88, wherein said first pitch is equal to said second pitch.

90. The cap of claim 88, wherein said first threads extend from said flange, and wherein said second threads are disposed on said circumferential wall.
